SEQUENCE LISTING (3 sequences)

SEQ ID NO: 1 - DNA, 6 bp, Homo sapiens
  misc_feature (1)..(6): Conserved telomeric repeat unit
ttaggg

SEQ ID NO: 2 - DNA, 45 bp, Artificial Sequence
  Synthetic primer
gttacaagattctcacacgctaaggctggaagccacaaggtaaac

SEQ ID NO: 3 - DNA, 45 bp, Artificial Sequence
  Synthetic primer
agttggcgaagcagtcgctagaagaacacccttttaagtctcagg

(19) United States
(12) Patent Application Publication
(10) Pub. No.: US 2004/0185477 A1
(43) Pub. Date: Sep. 23, 2004

(54) Methods of detecting differences in genomic sequence representation

(75) Inventor: Vladimir I. Slepnev, Newton, MA (US)

Correspondence Address: PALMER & DODGE, LLP, KATHLEEN M. WILLIAMS, 111 HUNTINGTON AVENUE, BOSTON, MA, 02199, US

(73) Assignee: Sention Inc.

(21) Appl. No.: 10/752,123

(22) Filed: Jan. 6, 2004

Related U.S. Application Data
(60) Provisional application No. 60/439,122, filed on Jan. 10, 2003.

(51) Int. Cl. C12Q 1/68; C12P 19/34

(52) U.S. Cl. 435/6; 435/91.2

(57) ABSTRACT

The invention relates to methods of genotyping the differential representation of one or more target genomic DNA sequences in a genomic DNA sample relative to a reference genomic DNA sample. The method uses tagged primer extension in which a set of tag sequences correspond to the identity of the elected target DNA sequence. Primer extension products are PCR amplified using a common set of tag-specific primers, the downstream primers bearing distinguishable labels. Following separation by size and/or charge, the detection of distinguishable label in a product of the anticipated size determines the identity and representation of the target DNA sequence in the sample. The method is well-suited for the genotyping of multiple target DNA sequence differences in one series of reactions.

### FIELD OF THE INVENTION

The invention relates to molecular genetic methods for the identification of sequence differences in the genome of an individual relative to the sequence of another individual or population of individuals. More particularly, the invention relates to methods for the identification of target DNA sequences whose representation can vary within genomic sequences.

### BACKGROUND OF THE INVENTION

Numerous diseases are thought to be initiated by disruptions in genomic stability. For example, sickle cell anemia, phenylketonuria, hemophilia, cystic fibrosis, and various cancers have been associated with one or more genetic mutation(s). Increased knowledge of the molecular basis for disease has lead to a proliferation of screening assays capable of detecting disease-associated nucleic acid mutations.

One such method identifies a genomic region thought to be associated with a disease and compares the wild-type sequence in that region with the sequence in a patient sample. Differences in the sequences constitute a positive screen. See e.g., Engelke, et al., Proc. Natl. Acad. Sci., 85: 544-548 (1988). Such methods are time-consuming, costly, and often result in an inability to identify the mutation of interest. Thus, sequencing is not practical for large-scale screening assays.

A variety of detection methods have been developed which exploit sequence variations in DNA using enzymatic and chemical cleavage techniques. A commonly-used screen for DNA polymorphisms consists of digesting DNA with restriction endonucleases and analyzing the resulting fragments by means of Southern blots, as reported by Botstein et al., Am. J. Hum. Genet., 32: 314-331 (1980) and White et al., Sci. Am., 258: 40-48 (1988). Mutations that affect the recognition sequence of the endonuclease will preclude enzymatic cleavage at that site, thereby altering the cleavage pattern of the DNA. Sequences are compared by looking for differences in restriction fragment lengths. A problem with this method (known as restriction fragment length polymorphism mapping or RFLP mapping) is its inability to detect mutations that do not affect cleavage with a restriction endonuclease.

A number of detection methods have also been developed which are based on template-dependent primer extension. Those methods can be placed into one of two categories: (1) methods using primers which span the region to be interrogated for the mutation, and (2) methods using primers which hybridize upstream of the region to be interrogated for the mutation.

In the first category, U.S. Pat. No. 5,578,458 reports a method in which single base mutations are detected by competitive oligonucleotide priming under hybridization conditions that favor the binding of a perfectly-matched primer as compared to one with a mismatch. U.S. Pat. No. 4,851,331 reports a similar method in which the 3′ terminal nucleotide of the primer corresponds to the variant nucleotide of interest. Since mismatching of the primer and the template at the 3′ terminal nucleotide of the primer inhibits elongation, significant differences in the amount of incorporation of a tracer nucleotide result under normal primer extension conditions.

Methods in the second category are based on incorporation of detectable, chain-terminating nucleotides in the extending primer. Such single nucleotide primer-guided extension assays have been used to detect aspartylglucosaminuria, hemophilia B, and cystic fibrosis; and for quantifying point mutations associated with Leber Hereditary Optic Neuropathy. See.e.g., Kuppuswamy et al., Proc. Natl. Acad. Sci. USA, 88: 1143-1147 (1991); Syvanen et al., Genomics, 8: 684-692 (1990); Juvonen et al., Human Genetics, 93: 16-20 (1994); Ikonen et al., PCR Meth. Applications, 1: 234-240 (1992); Ikonen et al., Proc. Natl. Acad. Sci. USA, 88: 11222-11226 (1991); Nikiforov et al., Nucleic Acids Research, 22: 4167-4175 (1994). An alternative primer extension method involving the addition of several nucleotides prior to the chain terminating nucleotide has also been proposed in order to enhance resolution of the extended primers based on their molecular weights. See e.g., Fahy et al., WO/96/30545 (1996).

Strategies based on primer extension require considerable optimization to ensure that only the perfectly annealed oligonucleotide functions as a primer for the extension reaction and often results in lower sensitivity due to a reduced yield of extended primer.

There is a need in the art for additional methods of detecting sequence differences. There is a particular need for methods that detect differences in genomic sequence representation, i.e., methods that detect sequence amplification, deletion, or rearrangement. An ideal method would detect differences in the genomic representation of sequences other than, or in addition to, single nucleotide differences.

### SUMMARY OF THE INVENTION

The present invention provides methods useful for genotyping nucleic acid samples with regard to one or more target DNA sequences. The methods of the invention use PCR amplification of primer extension products comprising heterologous sequence tags, which greatly increases the sensitivity and specificity of the assay, followed by capillary electrophoretic size separation, detection and sequencing of the amplified primer extension products. In one aspect, the size separation and product detection are performed in real time. Because the CE separation and detection techniques provide information including the amplified fragment size and the identity of label present on any given amplification product, the disclosed methods are particularly well suited for simultaneously analyzing samples for genotype with regard to variations in the representation of target DNA sequences. Each target DNA sequence can be detected by the amplification of a discretely sized amplification fragment bearing a distinguishably labeled sequence tag that specifically correlates with the presence of a target DNA sequence within the genomic DNA. Methods according to the invention also have the advantage of requiring one set of amplification primers for the detection of multiple target DNA sequences, thereby reducing the impact of problems related to the use of multiple different amplification primers.

In another aspect, the invention encompasses methods of identifying new target sequences for genomic region that differ in representation between individuals or between an individual and a larger population of individuals.

The invention encompasses a method of determining, for a given genomic DNA sample, a variation in the representation of an elected target DNA sequence within the sample, the method comprising the steps of:

a) producing a labeled, amplified DNA fragment by subjecting a population of primer extension products to an amplification regimen wherein the population of primer extension products is generated from a nucleic acid sample comprising genomic DNA, wherein the population of primer extension products comprises a population of nucleic acid molecules comprising a first, upstream tag sequence or the complement thereof, an elected target DNA sequence or the complement thereof, and a second, downstream tag sequence or the complement thereof, wherein the amplification regimen is performed using an upstream amplification primer comprising the upstream tag sequence, and a labeled downstream amplification primer comprising the downstream tag sequence; and

b) detecting a difference in the amount of signal from the resulting labeled amplified DNA fragment relative to a reference,

wherein the difference in the signal is indicative of a variation in the representation of the elected target DNA sequence within the genomic DNA sample.

In one embodiment, the label is a fluorescent label.

In another embodiment, step (b) comprises separating nucleic acid molecules made during the amplification regimen by size. In another embodiment, the separating comprises capillary electrophoresis.

In another embodiment, the amplification regimen comprises at least two amplification reaction cycles, wherein each cycle comprises polymerase extension of annealed primers. In another embodiment, the amplification regimen further comprises the steps, before the primer extension of annealed primers, of: 1) nucleic acid strand separation; and 2) oligonucleotide primer annealing. In another embodiment, the method further comprises the steps, during the amplification regimen and after at least one of the reaction cycles, of removing an aliquot of the amplification reaction, separating nucleic acid molecules by size and detecting the incorporation of the label wherein the detection determines the representation of the elected target DNA sequence within the genomic DNA sample.

In another embodiment, the method further comprises, before step (a), the steps of:

1) subjecting a genomic DNA sample to a primer extension reaction, wherein the primer extension reaction is performed using:

i) an upstream primer extension primer comprising the first, upstream tag sequence and a covalently linked hybridization region that can anneal to a sequence comprised by the elected DNA sequence; and

ii) a downstream primer extension primer comprising the second, downstream tag sequence and a covalently linked hybridization region that can specifically anneal to a sequence comprised by the elected target DNA sequence in the genomic DNA sample, wherein the hybridization region of the downstream primer anneals on the opposite strand from and downstream of the upstream primer on the elected target DNA sequence; and

2) repeating step (1) to generate a population of primer extension products comprising both an upstream primer sequence or the complement thereof and a downstream primer sequence or the complement thereof. In another embodiment, the method further comprises the step, after step (2), of removing unincorporated upstream and downstream primers.

In another embodiment, the method further comprises the step of sequencing the labeled amplified DNA fragment.

In another embodiment, the steps of removing, separating and detecting are performed after each cycle in the regimen.

In another embodiment, the separating comprises capillary electrophoresis.

In another embodiment, the method is performed in a modular apparatus comprising a thermal cycler, a sampling device, a capillary electrophoresis device and a fluorescence detector.

In another embodiment, the tag sequences comprise 15 to 40 nucleotides.

In another embodiment, the step of removing unincorporated upstream and downstream amplification primers comprises degrading the primers. In another embodiment, the degrading is performed using a heat labile exonuclease. In another embodiment, the heat labile exonuclease is selected from the group consisting of Exonuclease I and Exonuclease VII. In another embodiment, the heat labile exonuclease is thermally inactivated before continuing to step (d).

In another embodiment, either the upstream or downstream primer extension primer anneals to a repetitive DNA element.

In another embodiment, either the upstream or downstream primer extension primer anneals to a transposable DNA element.

In another embodiment, an increase of two fold or more in the signal, relative to a reference, is indicative of a variation in the representation of the elected target DNA sequence within the genomic DNA sample.

In another embodiment, a decrease of two fold or more in the signal, relative to a reference, is indicative of a variation in the representation of the elected target DNA sequence within the genomic DNA sample.

The invention further encompasses a method of determining, for a given genomic DNA sample, a variation in the representation of a group of elected target DNA sequences relative to a reference genomic DNA sample, the method comprising the steps of:

a) producing a set of labeled, amplified DNA fragments by subjecting a population of primer extension products generated from a nucleic acid sample comprising genomic DNA to an amplification regimen, wherein the population of primer extension products comprises a population of nucleic acid molecules comprising a common upstream tag sequence or the complement thereof, a member of the group of elected target DNA sequences, and a member of a set of downstream tag sequences or the complement thereof, wherein the amplification regimen is performed using:

i) an upstream amplification primer comprising the common upstream tag sequence; and

ii) a set of distinguishably labeled downstream amplification primers, each member of the set of labeled downstream amplification primers comprising a tag sequence comprised by a member of the set of downstream tag sequences, wherein each of the downstream tag sequences specifically corresponds to one member of the group of elected target DNA sequences; and

b) detecting a difference in the amount of signal from a labeled amplified fragment relative to the reference;

wherein a difference in the signal is indicative of a variation in the representation within the genomic DNA sample, of an elected target DNA sequence in the group of elected target DNA sequences.

In one embodiment, the method further comprises, before step (a), the steps of:

1) subjecting a genomic DNA sample to a primer extension reaction, wherein the primer extension reaction is performed using:

i) a set of upstream primer extension primers, each member of the set comprising a sequence that can anneal to a sequence comprised by a member of the group of elected DNA elected target DNA sequences and the common upstream tag sequence; and

ii) a set of downstream primer extension primers, each member of the set of downstream primer extension primers comprising a region that can anneal to a sequence comprised by a member of the group of elected target DNA sequences and one of the set of corresponding downstream tag sequences, wherein the region that can anneal to a sequence comprised by a member of the group of elected target DNA sequences anneals downstream of and on the opposite strand from a member of the set of upstream primers;

2) repeating step (1) to generate a population of primer extension products comprising both an upstream primer sequence or the complement thereof and a downstream primer sequence or the complement thereof. In another embodiment, the method further comprises the step, after step (2), of removing unincorporated upstream and downstream primer extension primers.

In another embodiment, the distinguishable label is a fluorescent label.

In another embodiment, step (b) comprises separating nucleic acid molecules made during the amplification regimen by size. In another embodiment, the separating comprises capillary electrophoresis.

In another embodiment, the amplification regimen comprises at least two amplification reaction cycles, wherein each cycle comprises the step of polymerase extension of annealed primers. In another embodiment, the amplification regimen further comprises, before the polymerase extension step, the steps of: 1) nucleic acid strand separation; and 2) oligonucleotide primer annealing.

In another embodiment, the method further comprises the steps, during the amplification regimen and after at least one of the reaction cycles, of removing an aliquot of the amplification reaction, separating nucleic acid molecules by size, and detecting the incorporation of a distinguishable label. In another embodiment, the removing, separating and detecting are performed after each cycle in the regimen. In another embodiment, the separating comprises capillary electrophoresis.

In another embodiment, the method is performed in a modular apparatus comprising a thermal cycler, a sampling device, a capillary electrophoresis device and a fluorescence detector.

In another embodiment, the tag sequences comprise 15 to 40 nucleotides.

In another embodiment, the step of removing unincorporated upstream and downstream amplification primers comprises degrading the primers. In another embodiment, the degrading is performed using a heat labile exonuclease. In another embodiment, the heat labile exonuclease is selected from the group consisting of Exonuclease I and Exonuclease VII.

In another embodiment, the heat labile exonuclease is thermally inactivated before continuing to step (d).

In another embodiment, either the upstream or downstream primer extension primer anneals to a repetitive DNA element.

In another embodiment, either the upstream or downstream primer extension primer anneals to a transposable DNA element.

In another embodiment, an increase of two fold or more in the signal, relative to a reference, is indicative of the variation in the representation of the elected target DNA sequence within the genomic DNA sample.

In another embodiment, a decrease of two fold or more in the signal, relative to a reference, is indicative of the variation in the representation of the elected target DNA sequence within the genomic DNA sample.

In another embodiment, each upstream primer extension primer anneals to a sequence, comprised by a member of the group of elected target DNA sequences, that is located at a distance from a downstream primer extension primer that is characteristic for said member of the group of elected target DNA sequences.

The invention further encompasses a method of determining, for a given genomic DNA sample, a variation in the representation of an arbitrary genomic DNA sequence to be interrogated within the sample, the method comprising the steps of:

a) producing a labeled, amplified DNA fragment by subjecting a population of primer extension products generated from a nucleic acid sample comprising genomic DNA to an amplification regimen, wherein the population of primer extension products comprises a population of nucleic acid molecules, each member of the population of nucleic acid molecules comprising an upstream tag sequence or the complement thereof, an arbitrary genomic DNA sequence and a downstream tag sequence or the complement thereof, wherein the amplification regimen is performed using:

i) an upstream amplification primer comprising the upstream tag sequence; and

ii) a distinguishably labeled downstream amplification primer comprising the downstream tag sequence; and

b) detecting a difference in the signal from the resulting labeled amplified DNA fragment relative to a reference;

wherein the difference in the signal is indicative of a variation in the representation of the arbitrary genomic DNA sequence within the genomic DNA sample.

In one embodiment, the method further comprises the steps, before step (a), of:

1) subjecting a sample comprising genomic DNA to a primer extension reaction, wherein the primer extension reaction is performed using:

i) an upstream primer extension primer comprising a first arbitrary DNA sequence and the upstream tag sequence; and

ii) a downstream primer extension primer comprising a second arbitrary DNA sequence and the downstream tag sequence; and

2) repeating step (1) to generate a population of primer extension products comprising both an upstream tag sequence or the complement thereof and a downstream tag sequence or the complement thereof. In another embodiment, the method further comprises the step, after step (2), of removing unincorporated upstream and downstream primer extension primers.

In another embodiment, the label is a fluorescent label.

In another embodiment, step (b) comprises separating nucleic acid molecules made during the amplification regimen by size and/or by charge. In another embodiment, the separating comprises capillary electrophoresis.

In another embodiment, the amplification regimen comprises at least two amplification reaction cycles, wherein each cycle comprises the step of polymerase extension of annealed primers. In another embodiment, the amplification regimen further comprises, before the step of polymerase extension of annealed primers, the steps of: i) nucleic acid strand separation; and ii) oligonucleotide primer annealing.

In another embodiment, the method further comprises the steps, during the amplification regimen and after at least one of the reaction cycles, of removing an aliquot of the amplification reaction, separating nucleic acid molecules by size, and detecting the incorporation of a distinguishable label. In another embodiment, the method further comprises the step, after the step of detecting the incorporation of a distinguishable label, of sequencing the resulting amplified genomic DNA, wherein the sequencing determines the identity of the elected DNA sequence. In another embodiment, the removing, separating and detecting are performed after each cycle in the regimen. In another embodiment, the separating comprises capillary electrophoresis.

In another embodiment, the method is performed in a modular apparatus comprising a thermal cycler, a sampling device, a capillary electrophoresis device and a fluorescence detector.

In another embodiment, the tag sequence comprises 15 to 40 nucleotides.

In another embodiment, the step of removing unincorporated upstream and downstream amplification primers comprises degrading the primers. In another embodiment, the degrading is performed using a heat labile exonuclease. In another embodiment, the heat labile exonuclease is selected from the group consisting of Exonuclease I and Exonuclease VII. In another embodiment, the heat labile exonuclease is thermally inactivated after degrading the upstream and downstream primer extension primers.

In another embodiment, an increase of two fold or more in the signal, relative to a reference, is indicative of the variation in the representation of the arbitrary target DNA sequence within the genomic DNA sample.

In another embodiment, a decrease of two fold or more in the signal, relative to a reference, is indicative of the variation in the representation of the arbitrary target DNA sequence within said genomic DNA sample.

The invention further encompasses a method of determining, for a given genomic DNA sample, a variation in the representation of one or more of a group of arbitrary genomic DNA sequences relative to a reference genomic DNA sample, the method comprising the steps of:

a) producing a set of labeled, amplified DNA fragments by subjecting a population of primer extension products generated from a sample comprising genomic DNA to an amplification regimen, wherein the population of primer extension products comprises a population of nucleic acid molecules comprising a common upstream tag sequence or the complement thereof, an arbitrary genomic DNA sequence, and a member of a set of downstream tag sequences or the complement thereof, wherein the amplification regimen is performed using:

i) an upstream amplification primer comprising the common upstream tag sequence; and

ii) a set of distinguishably labeled downstream amplification primers, each member of the set of labeled downstream amplification primers comprising a tag sequence comprised by a member of the set of downstream tag sequences; and

b) detecting a difference in the signal from a resulting labeled amplified fragment relative to the reference;

wherein a difference in the signal is indicative of a variation in the representation of an elected target DNA sequence, selected from the group of arbitrary target DNA sequences, within the genomic DNA sample.

In one embodiment, the method further comprises the steps, before step (a), of:

1) subjecting a genomic DNA sample to a primer extension reaction, wherein the primer extension reaction is performed using:

i) a set of upstream primer extension primers, each member of the set comprising the common upstream tag sequence and a region that can anneal to a member of the group of arbitrary genomic DNA sequences; and

ii) a set of downstream primer extension primers, each member of the set of downstream primer extension primers comprising a region that can anneal to a sequence comprised by a member of the group of arbitrary genomic DNA sequences and one of the set of downstream tag sequences, wherein the region than can anneal to a sequence comprised by a member of the group of arbitrary target DNA sequences anneals downstream of and on the opposite strand from a member of the set of upstream primer extension primers; and

b) repeating step (a) to generate a population of primer extension products comprising both an upstream tag sequence or the complement thereof and a downstream tag sequence or the complement thereof. In another embodiment, the method further includes the step, after step (b) or removing unincorporated upstream and downstream primer extension primers.

In another embodiment, the distinguishable label is a fluorescent label.

In another embodiment, step (b) comprises separating nucleic acid molecules made during the amplification regimen by size. In another embodiment, the separating comprises capillary electrophoresis.

In another embodiment, the amplification regimen comprises at least two amplification reaction cycles, wherein each cycle comprises the step of polymerase extension of annealed primers. In another embodiment, the amplification regimen further comprises the steps, before the step of polymerase extension of annealed primers, of i) nucleic acid strand separation and ii) oligonucleotide primer annealing.

In another embodiment, the method further comprises the steps, during the amplification regimen and after at least one of the reaction cycles, of removing an aliquot of the amplification reaction, separating nucleic acid molecules by size, and detecting the incorporation of a distinguishable label, and sequencing the amplified genomic DNA wherein the sequencing determines the identity of the arbitrary DNA sequence. In another embodiment, The the removing, separating and detecting are performed after each cycle in the regimen. In another embodiment, the method further comprises the step, after the detecting, of sequencing the resulting amplified genomic DNA, wherein the sequencing determines the identity of an arbitrary DNA sequence. In another embodiment, the separating comprises capillary electrophoresis.

In another embodiment, the method is performed in a modular apparatus comprising a thermal cycler, a sampling device, a capillary electrophoresis device and a fluorescence detector.

In another embodiment, the tag sequence comprises 15 to 40 nucleotides.

In another embodiment, the step of removing unincorporated upstream and downstream amplification primers comprises degrading the primers. In another embodiment, the degrading is performed using a heat labile exonuclease. In another embodiment, the heat labile exonuclease is selected from the group consisting of Exonuclease I and Exonuclease VII. In another embodiment, the heat labile exonuclease is thermally inactivated after the degrading.

In another embodiment, an increase of two fold or more in the signal, relative to a reference, is indicative of the variation in the representation of the arbitrary target DNA sequence within the genomic DNA sample.

In another embodiment, a decrease of two fold or more in the signal, relative to a reference, is indicative of the variation in the representation of the arbitrary target DNA sequence within the genomic DNA sample.

In another embodiment, each upstream primer extension primer anneals to a sequence, comprised by a member of the group of arbitrary DNA e sequences, that is located at a distance from the set of downstream primer extension primers that is characteristic for the upstream primer extension primer and the downstream primer extension primer.

The invention further encompasses a kit for the determination of the variation in representation of an elected target DNA sequence within a genomic DNA sample, the kit comprising:

a) an upstream primer extension primer comprising an upstream tag sequence and a covalently linked hybridization region that can anneal to a sequence at a known distance upstream of the elected target DNA sequence; and

b) a downstream primer extension primer comprising a downstream tag sequence and a covalently linked hybridization region that can anneal to an elected target DNA sequence within the genomic DNA sample.

In one embodiment, the kit further comprises an upstream tag-specific amplification primer and a labeled downstream tag-specific amplification primer.

As used herein, the term “sample” refers to a biological material which is isolated from its natural environment and contains a polynucleotide, preferably genomic DNA. A “sample” according to the invention can consist of purified or isolated polynucleotide, or it may comprise a biological sample such as a tissue sample, a biological fluid sample, or a cell sample comprising a polynucleotide. A biological fluid includes blood, plasma, sputum, urine, cerebrospinal fluid, lavages, and leukophoresis samples. A sample of the present invention may be any plant, animal, bacterial or viral material containing a polynucleotide.

As used herein, “genomic DNA” refers to chromosomal DNA, as opposed to complementary DNA copied from an RNA transcript. “Genomic DNA sample”, as used herein, may be all or a portion of the genomic DNA present in a sample as defined herein.

As used herein, the term “polymorphism” or simply “sequence difference” refers to a nucleic acid sequence variation. When compared to a naturally occurring sequence, a polymorphism can be present at a frequency of greater than 0.01%, 0.1%, 1% or greater in a population. As used herein, a polymorphism can be an insertion, deletion, duplication, or rearrangement. A polymorphism can be phenotypically neutral or can have an associated variant phenotype that distinguishes it from that exhibited by the predominant sequence at that locus. As used herein, “neutral polymorphism” refers to a polymorphism in which the sequence variation does not alter gene function, and “mutation” “functional polymorphism” or “functional sequence difference” refers to a sequence variation which does alter gene function, and which thus has an associated phenotype.

As used herein, the term “representation” refers to the degree to which a given genomic sequence is present in a given individual's genome. This degree refers to the presence or absence of a given sequence, as well as to the number of copies of a given sequence present in a genome. In the simplest format, when comparing the representation of one sequence between two individuals or between one individual and a population of individuals, “representation” can be expressed as the number of copies of the sequence present in each individual or in the individuals of a majority or other chosen proportion of the population (a “reference” population). “Representation” can also be expressed as the relative ratio of the number of copies of the sequence between two individuals or between an individual and the number of copies of the sequence present in a majority or other chosen proportion of the population.

As used herein, the term “variation in representation” refers to a difference in the number or presence of a given genomic DNA sequence in one individual relative to the presence or number of that sequence in a reference genome (e.g., that of another individual or population of individuals). A “variation in representation” is a difference of at least one copy of (more than or less than) the given sequence relative to the reference, but can encompass differences of, for example, 2 copies, 5 copies, 10 copies, 50 copies, 100 copies, 200 copies or more.

As used herein, “differentially represented sequence” refers to a genomic DNA sequence that either occurs more frequently (i.e., at least one more copy) or less frequently (i.e., at least one copy less frequently, including the complete absence of the sequence) in a test genomic DNA sample relative to the frequency of that sequence in a reference genomic DNA sample, as the term is defined herein. A sequence that occurs less frequently in one individual relative to another can be said to be “underrepresented” relative to the other, while a sequence that occurs more frequently can be said to be “overrepresented” relative to the other.

As used herein, the term “population” refers to a collection of at least two of a given item or type of item. The term “population” as applied to a number of individuals, e.g., humans, means at least two such individuals, but can encompass collections of tens, hundreds, thousands or more individuals falling within the given group. The members of a population have at least one common characteristic that qualifies them as members of that group, whether it be simply the identity of the item (e.g., humans) or one or more further specific characteristics (e.g., humans living or born in a given area, humans having or lacking a given phenotypic characteristic, disease or disorder, etc.). The term “population” as applied to a collection of nucleic acid molecules means at least two such molecules, most often more (e.g., 10, 10², 10³, 10⁴, 10⁵, 10⁶, 10⁷, 10⁸, 10⁹, 10¹⁰ or more). Without limitation, the common characteristic for a population of nucleic acid molecules can be that they were generated from the same genomic DNA sample, that they are of the same or similar size, that they have the same or similar sequence, that they comprise a given tag sequence, or even that they do not comprise a given tag sequence.

As used herein, the term “generated from,” when used in relation to a nucleic acid in general or to a primer extension product in particular, means that the nucleic acid comprises a copy, either an exact copy or a complementary copy, of the nucleic acid it is said to be “generated” from. As an example, primer extension products are “generated from” genomic DNA by a polymerase that uses one strand of the genomic DNA as a template for the incorporation of sequence complementary to the template strand onto an annealed primer.

When referring to the genotype of an individual with regard to a “polymorphism” or “sequence difference”, the “predominant allele” is that which occurs most frequently in the population being examined (i.e., when there are two alleles, the allele that occurs in greater than 50% of the population is the predominant allele; when there are more than two alleles, the “predominant allele” is that which occurs in the subject population at the highest frequency, e.g., at least 5% higher frequency, relative to the other alleles at that site). The term “variant allele” is used to refer to the allele or alleles occurring less frequently than the predominant allele in that population (e.g., when there are two alleles, the variant allele is that which occurs in less than 50% of the subject population; when there are more than two alleles, the variant alleles are all of those that occur less frequently, e.g., at least 5% less frequently, than the predominant allele).

As used herein, an “oligonucleotide primer” or primer refers to a polynucleotide molecule (i.e., DNA or RNA) capable of annealing to a polynucleotide template and providing a 3′ end to produce an enzymatic extension product which is complementary to the polynucleotide template. The conditions for initiation and extension usually include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer according to the invention can be single- or double-stranded. The primer is single-stranded for maximum efficiency in amplification, and the primer and its complement form a double-stranded polynucleotide. “Primers” useful in the present invention are less than or equal to 100 nucleotides in length, e.g., less than or equal to 90, or 80, or 70, or 60, or 50, or 40, or 30, or 20, or 15, or equal to 10 nucleotides in length.

As used herein, the term “primer extension” means the template-dependent incorporation of at least one complementary nucleotide, by a nucleic acid polymerase, onto the 3′ end of an annealed primer. Polymerase extension preferably adds more than one nucleotide, preferably up to and including nucleotides corresponding to the fill length of the template. Conditions for polymerase extension vary with the identity of the polymerase. The temperature of polymerase extension is based upon the known activity properties of the enzyme. In general, although the enzymes retain at least partial activity below their optimal extension temperatures, polymerase extension by the most commonly used thermostable polymerases (e.g., Taq polymerase and variants thereof) is performed at 65° C. to 75° C., preferably about 68-72° C.

As used herein, the term “primer extension reaction” refers to nucleic acid molecules generated by the process of polymerase extension.

As used herein, the term “tag sequence,” or simply “tag” refers to a nucleotide sequence, preferably a heterologous or artificial nucleotide sequence, that is attached to an oligonucleotide primer via standard phosphodiester linkage (i.e., phosphodiester linkage between the 3′ OH of the tag and the 5′ phosphate of the oligonucleotide) and permits the identification or tracing of polynucleotides into which the “tag” is incorporated (incorporated for example, by primer extension or amplification of a primer extension product). A “tag” sequence according to the invention will comprise at least 15, and preferably 20 to 30 nucleotides and will preferably not hybridize under primer extension conditions to a sequence in the genome of the organism being genotyped. A tag sequence according to the invention can be, but is not necessarily, random.

When used in reference to a nucleic acid primer or tag sequence, the term “arbitrary” means that the sequence was initially selected only on the basis of its length. That is, the sequence was randomly selected; factors such as G+C content or similarity to other sequences were not criteria in the initial selection of the sequence. One may subsequently choose to use a given “arbitrary” sequence, initially selected only on the basis of its length, for use as a primer or tag based on its G+C content or lack of similarity to known sequences, yet the sequence remains “arbitrary” as the term is used herein because its initial selection was performed only on the basis of its length.

As used herein, the term “specifically corresponds” means that a given nucleic acid tag sequence on an oligonucleotide is only used with a given elected DNA target sequence, such that the presence of the tag sequence in a primer extension product or amplification product is indicative of the presence of that elected DNA target sequence (or its complement).

As used herein, the term “amplification regimen” refers to a process of specifically amplifying, i.e., increasing the abundance of, a nucleic acid sequence of interest. An amplification regimen according to the invention comprises at least two, and preferably at least 5, 10, 15, 20, 25, 30, 35 or more iterative cycles, where each cycle comprises the steps of: 1) strand separation (e.g., thermal denaturation); 2) oligonucleotide primer annealing to template molecules; and 3) nucleic acid polymerase extension of the annealed primers. Conditions and times necessary for each of these steps are well known in the art. Amplification achieved using an amplification regimen is preferably exponential, but can alternatively be linear. An amplification regimen according to the invention is preferably performed in a thermal cycler, many of which are commercially available.

As used herein, the term “set” means a group of nucleic acid samples, primers or other entities. A set will comprise a known number of, and at least two of such entities.

As used herein, the relative terms “upstream” and “downstream” are used to refer to positions of a polynucleotide relative to an elected DNA target sequence. Generally, “upstream” refers to 5′ of the elected DNA target sequence, and “downstream” refers to 3′ of the elected DNA target sequence. It is understood that the choice of “upstream” and “downstream” in a double-stranded DNA sequence is largely arbitrary, in that one may choose to focus on either strand, and the direction that is “upstream” or “downstream” of the elected DNA target sequence will change, depending upon which strand is chosen as the “reference” strand. In order to avoid any ambiguity, as used herein to describe a given method, the “reference” strand for the selection of the terms “upstream” and “downstream” will remain the same throughout that method.

As used herein, the term “distinguishably labeled” means that the signal from one labeled oligonucleotide primer or a nucleic acid molecule into which it is incorporated can be distinguished from the signal from another such labeled primer or nucleic acid molecule. Detectable labels can comprise, for example, a light-absorbing dye, a fluorescent dye, or a radioactive label. Fluorescent dyes are preferred. Generally, a fluorescent signal is distinguishable from another fluorescent signal if the peak emission wavelengths are separated by at least 20 nm. Greater peak separation is preferred, especially where the emission peaks of fluorophores in a given reaction are wide, as opposed to narrow or more abrupt peaks.

As used herein, the term “separating nucleic acid molecules” refers to the process of physically separating nucleic acid molecules in a sample or aliquot on the basis of size and/or charge. As used herein, separating nucleic acid molecules “by size” encompasses electrophoretic separation—while charge is important in electrophoretic separation, all nucleic acids are negatively charged under standard conditions, such that size is the predominant factor in such separation. When separating nucleic acid molecules is called for, electrophoretic separation is preferred, and capillary electrophoretic separation is most preferred.

As used herein, the term “detecting the incorporation” refers to the process of determining whether a given labeled oligonucleotide primer has been extended, thereby incorporating the label into the primer extension or amplification product. Detection can be by any means compatible with the detectable label, but will preferably involve detection of a fluorescent label. Detecting encompasses determination of both the presence and the abundance of label in a primer extension or amplification product. Fluorescence detectors are well known in the art.

As used herein, the term “specifically anneal” or “specifically hybridize” means that under given hybridization conditions a probe or primer hybridizes only to a target sequence in a sample comprising the target sequence. Given hybridization conditions include the conditions for the annealing step in an amplification regimen, i.e., annealing temperature selected on the basis of predicted Tₘ, and salt conditions suitable for the polymerase enzyme of choice.

As used herein, the term “strand separation” or “separating the strands” means treatment of a nucleic acid sample such that complementary double-stranded molecules are separated into two single strands available for annealing to an oligonucleotide primer. Strand separation according to the invention is achieved by heating the nucleic acid sample above its Tₘ. Generally, for a sample containing nucleic acid molecules in buffer suitable for a nucleic acid polymerase, heating to 94° C. is sufficient to achieve strand separation according to the invention. An exemplary buffer contains 50 mM KCl, 10 mM Tric-HCl (pH 8.8@25° C.), 0.5 to 3 mM MgCl₂, and 0.1% BSA.

As used herein, the term “anneal” means permitting oligonucleotide primers to hybridize to template nucleic acid strands. Conditions for primer annealing vary with the length and sequence of the primer and are based upon the calculated Tₘ for the primer. Generally, an annealing step in an amplification regimen involves reducing the temperature following the strand separation step to a temperature based on the calculated Tₘ for the primer sequence, for a time sufficient to permit such annealing. Tₘ can be readily predicted by one of skill in the art using any of a number of widely available algorithms (e.g., Oligo™, Primer Design and programs available on the internet, including Primer3 and Oligo Calculator). For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can temperatures more than 5° C. below or above the predicted Tₘ (e.g., 6° C. below, 8° C. below, 10° C. below or lower and 6° C. above, 8° C. above, or 10° C. above). Generally, the closer the annealing temperature is to the Tₘ, the more specific is the annealing. Time of primer annealing depends largely upon the volume of the reaction, with larger volumes requiring longer times, but also depends upon primer and template concentrations, with higher relative concentrations of primer to template requiring less time than lower. Depending upon volume and relative primer/template concentration, primer annealing steps in an amplification regimen can be on the order of 1 second to 5 minutes, but will generally be between 10 seconds and 2 minutes, preferably on the order of 30 seconds to 2 minutes.

As used herein, the term “region that can anneal to a sequence at a known distance upstream of an elected DNA target sequence” refers to a sequence of nucleotides, located at the 3′ end of an oligonucleotide, that specifically hybridizes to a sequence upstream (i.e., 5′) of a known target DNA sequence being genotyped in a sample of nucleic acid. The “3′ region that hybridizes” will be at least 12 nucleotides long, and preferably at least 15, 18, 21, 24, 27, 30 nucleotides or more. The “region that can anneal” is selected to be a known distance from the target DNA sequence so as to give rise to an amplification product that is distinctly sized relative to other amplification products in a method according to the invention. The “known distance” can be from 50 to 1000 nucleotides, and is preferably from 50 to 500 nucleotides or 50 to 250 nucleotides.

As used herein, the term “complementary” refers to the hierarchy of hydrogen-bonded base pair formation preferences between the four deoxyribonucleotides G, A, T, and C, such that A pairs with T and G pairs with C.

As used herein, the phrase “nucleic acid polymerase” refers an enzyme that catalyzes the template-dependent polymerization of nucleoside triphosphates to form primer extension products that are complementary to one of the nucleic acid strands of the template nucleic acid sequence. A nucleic acid polymerase enzyme initiates synthesis at the 3′ end of an annealed primer and proceeds in the direction toward the 5′ end of the template. Numerous nucleic acid polymerases are known in the art and commercially available. One group of preferred nucleic acid polymerases are thermostable, i.e., they retain function after being subjected to temperatures sufficient to denature annealed strands of complementary nucleic acids.

As used herein, the term “aliquot” refers to a sample of an amplification reaction taken during the cycling regimen. An aliquot is less than the total volume of the reaction, and is preferably 0.1-30% in volume. In one embodiment of the invention, for each aliquot removed, an equal volume of reaction buffer containing reagents necessary for the reaction (e.g., buffer, salt, nucleotides, and polymerase enzyme) is introduced.

As used herein, the term “real time” means that the measurement of the accumulation of products in a nucleic acid amplification reaction is at least initiated, and preferably completed during or concurrent with the amplification regimen. Thus, for the measurement process to be considered “real time”, at least the initiation of the measurement or detection of amplification products in each aliquot is concurrent with the amplification process. By “initiated” is meant that an aliquot is withdrawn and placed into a separation apparatus, e.g., a capillary electrophoresis capillary, and separation is begun. The completion of the measurement is the detection of labeled species in the separated nucleic acids from the aliquot. Because the time necessary for separation and detection may exceed the time of each individual cycle of the amplification regimen, there may be a lag in the detection of the amplification products of up to 120 minutes beyond the completion of the amplification regimen. Preferably such lag or delay is less than 30 minutes, e.g., 25 minutes, 20 minutes, 15 minutes, 10 minutes, 5 minutes, 4 minutes, 3 minutes, 2 minutes, 1 minute or less, including no lag or delay.

As used herein, the term “capillary electrophoresis” means the electrophoretic separation of nucleic acid molecules in an aliquot from an amplification reaction wherein the separation is performed in a capillary tube. Capillary tubes are available with inner diameters from about 10 to 300 μm, and can range from about 0.2 cm to about 3 m in length, but are preferably in the range of 0.5 cm to 20 cm, more preferably in the range of 0.5 cm to 10 cm. In addition, the use of microfluidic microcapillaries (available, e.g., from Caliper or Agilent Technologies) is specifically contemplated within the meaning of “capillary electrophoresis.”

As used herein, the term “modular apparatus” means an apparatus that comprises individual units in which certain processes of the methods according to the invention are performed. The individual units of a modular apparatus can be but are not necessarily physically connected, but it is preferred that the individual units are controlled by a central control device such as a computer. An example of a modular apparatus useful according to the invention has a thermal cycler unit, a sampler unit, and a capillary electrophoresis unit with a fluorescence detector. The modular apparatus useful according to the invention can also comprise a robotic arm to transfer samples from the cycling reaction to the electrophoresis unit.

As used herein, the term “sampling device” refers to a mechanism that withdraws an aliquot from an amplification during the amplification regimen. Sampling devices useful according to the invention will preferably be adapted to minimize contamination of the cycling reaction(s), by, for example, using pipeting tips or needles that are either disposed of after a single sample is withdrawn, or by incorporating one or more steps of washing the needle or tip after each sample is withdrawn. Alternatively, the sampling device can contact the capillary to be used for capillary electrophoresis directly with the amplification reaction in order to load an aliquot into the capillary. Alternatively, the sample device can include a fluidic line (e.g. a tube) connected to the controllable valve which will open at particular cycle. Sampling devices known in the art include, for example, the multipurpose Robbins Scientific Hydra 96 pipettor, which is adapted to sampling to or from 96 well plates. This and others can be readily adapted for use according to the methods of the invention.

As used herein, the term “robotic arm” means a device, preferably controlled by a microprocessor that physically transfers samples, tubes, or plates containing samples from one location to another. Each location can be a unit in a modular apparatus useful according to the invention. An example of a robotic arm useful according to the invention is the Mitsubishi RV-E2 Robotic Arm. Software for the control of robotic arms is generally available from the manufacturer of the arm.

As used herein, “real time QPCR (‘quantitative PCR’)” refers to the direct monitoring of the progress of a PCR amplification as it is occurring without the need for repeated sampling of the reaction products. In real-time QPCR, the reaction products are monitored as they are generated and are tracked after they rise above background but before the reaction reaches a plateau. The number of cycles required to achieve a chosen level of fluorescence varies directly with the concentration of amplifiable targets at the beginning of the PCR process, enabling a measure of fluorescent intensity to provide a measure of the amount of target DNA in a sample in real time.

Holland et al. (1991, Proc. Natl. Acad. Sci. U.S.A. 88: 7276-7280), U.S. Pat. No. 5,210,015 and others have disclosed fluorescence-based approaches to provide real time measurements of amplification products during QPCR. In the Taq-Man approach, an oligonucleotide probe is used that contains a reporter molecule—quencher molecule pair that specifically anneals to a region of a target polynucleotide “downstream”, i.e. in the direction of extension of primer binding sites. The reporter molecule and quencher molecule are positioned on the probe sufficiently close to each other such that whenever the reporter molecule is excited, the energy of the excited state nonradiatively transfers to the quencher molecule where it either dissipates nonradiatively or is emitted at a different emission frequency than that of the reporter molecule. During strand extension by a DNA polymerase, the probe anneals to the template where it is digested by the 5′ to 3′ exonuclease activity of the polymerase. As a result of the probe being digested, the reporter molecule is effectively separated from the quencher molecule such that the quencher molecule is no longer close enough to the reporter molecule to quench the reporter molecule's fluorescence. Thus, as more and more probes are digested during amplification, the number of reporter molecules in solution increases, thus resulting in an increasing number of unquenched reporter molecules which produce a stronger and stronger fluorescent signal.

The other most commonly used real time QPCR approach uses the so-called “molecular beacons” technology. This approach is also based upon the presence of a quencher-fluorophore pair on an oligonucleotide probe. In the beacon approach, a probe is designed with a stem-loop structure, and the two ends of the molecule are labeled with a fluorophore and a quencher of that fluorophore, respectively. In the absence of target polynucleotide, the complementary sequences on either end of the molecule permit stem formation, bringing the labeled ends of the molecule together, so that fluorescence from the fluorophore is quenched. In the presence of the target polynucleotide, which bears sequence complementary to the loop and part of the stem structure of the beacon probe, the intermolecular hybridization of the probe to the target is energetically favored over intramolecular stem-loop formation, resulting in the separation of the fluorophore and the quencher, so that fluorescent signal is emitted upon excitation of the fluorophore. The more target present, the more probe hybridizes to it, and the more fluorophore is freed from quenching, providing a read out of the amplification process in real time.

In one embodiment of the invention, concurrent sampling of the amplification reaction after each cycle of the amplification regimen together with real time QPCR surveillance is envisioned to ensure accurate identification and quantification of the amplified DNA fragment simultaneously in both test and reference genomic DNA samples.

As used herein, the term “amplified fragment” refers to polynucleotides which are copies of a portion of a particular polynucleotide sequence and/or its complementary sequence, which correspond in nucleotide sequence to the template polynucleotide sequence and its complementary sequence. An “amplified product,” according to the invention, can be DNA or RNA, and it can be double-stranded or single-stranded.

As used herein, the term “distinctly sized amplification product” means an amplification product that is resolvable from amplification products of different sizes. “Different sizes” refers to nucleic acid molecules that differ by at least one nucleotide in length. Generally, distinctly sized amplification products useful according to the invention differ by greater than or equal to more nucleotides than the limit of resolution for the separation process used in a given method according to the invention. For example, when the limit of resolution of separation is one base, distinctly sized amplification products differ by at least one base in length, but can differ by 2 bases, 5 bases, 10 bases, 20 bases, 50 bases, 100 bases or more. When the limit of resolution is, for example, 10 bases, distinctly sized amplification products will differ by at least 10 bases, but can differ by 11 bases, 15 bases, 20 bases, 30 bases, 50 bases, 100 bases or more.

As used herein, the term “profile” or the equivalent terms “amplification curve” and “amplification plot” mean a mathematical curve representing the signal from a detectable label incorporated into a nucleic acid sequence of interest at two or more steps in an amplification regimen, plotted as a function of the cycle number from which the samples were withdrawn. The profile is preferably generated by plotting the fluorescence of each band detected after capillary electrophoresis separation of nucleic acids in the individual reaction samples. Most commercially available fluorescence detectors are interfaced with software permitting the generation of curves based on the signal detected.

The number of differentially represented sequences that could be investigated in a single reaction can be estimated based on the measurable difference of the product size (1-2 bases) and on the separable size of PCR products (500-1000 bp) and can be as high as 1000, but is preferably 100-200.

As used herein, the term “heat-labile exonuclease” refers to an enzyme that degrades single-stranded nucleic acid molecules or overhanging single strands on partially double stranded nucleic acid molecules and is irreversibly inactivated by incubation at an elevated temperature. The temperature for inactivation will vary with the enzyme and with, for example, buffer conditions and enzyme concentration. Conditions for enzyme inactivation are known to those skilled in the art. A non-limiting example of a heat-labile exonuclease useful according to the invention is Exonuclease I (ExoI), from E. coli (commercially available from, e.g., New England Biolabs, Beverly Mass.). Exol is inactivated by incubation at 80° C. for 20 minutes.

As used herein, the term “substantially lacking sequence specific for a gene in the genome of the organism” means that a given primer will not generate a primer extension product when incubated under primer extension conditions with genomic DNA from the organism being investigated with respect to differences in sequence representation.

As used herein, an “elected target DNA sequence” refers to any DNA sequence whose representation within a genomic DNA sample may vary with respect to a reference. In a preferred embodiment, the elected DNA target sequence is that of an element known or suspected to vary in genomic representation between individuals. In another preferred embodiment, the elected DNA target sequence is a repetitive DNA element. In another preferred embodiment, the elected DNA target sequence is a rearranged DNA element. In another preferred embodiment, the elected DNA target sequence is an arbitrary DNA sequence. A “target DNA sequence”, according to the invention, is at least 10, 11, 12, 13, 14, 15, 20 or 25 or more nucleotides in length.

As used herein, a “rearranged DNA sequence” refers to any DNA sequence that is deleted, duplicated, amplified, or inserted with respect to the wild type genomic DNA sequence. According to the invention, a DNA sequence is said to be rearranged if 1 nucleotide, 5 nucleotides, 10 nucleotides, 50 nucleotides, 100 nucleotides, 500 nucleotides, 1000 nucleotides, 5000 nucleotides, 10,000 nucleotides, 25,000 nucleotides or more are deleted, duplicated, amplified, or inserted with respect to the wild type genomic DNA sequence. In one embodiment, a “rearranged DNA sequence” refers to the insertion of transposable elements within genomic DNA (for example LINEs or SINEs; reviewed by Prak, E. T. and Kazazian, H. H. Nature Genetics Reviews (2000), 1, 134-144).

As used herein, the term “wild-type genomic DNA sequence” refers to that sequence, over a given genomic region that is present in a majority of individuals in a given population.

As used herein, a “repetitive DNA sequence” refers to non-coding sequences of DNA containing short repeated sequences that are dispersed randomly in the genome. For example, a well known repetitive DNA element is the Alu DNA repeat element, a million copies of which can be found within the human genome.

As used herein, the term “arbitrary” refers to DNA sequences that are randomly selected.

As used herein, a “genomic DNA sample” or “test genomic DNA sample” refers to a sample taken from a patient to be tested for the differential representation of target genomic DNA sequences relative to one or more reference genomic DNA samples. In a preferred embodiment, test genomic DNA samples are taken from patients having or suspected of having a particular phenotype or disease, including but not limited to patients afflicted with neurological disease, cancer, diabetes, and other inherited diseases.

As used herein, a reference genomic DNA sample refers to a sample taken from an individual that is randomly selected within the population and who does not exhibit any detectable phenotype or disease. In a preferred embodiment, the invention provides for at least two, preferably five reference genomic DNA samples from individuals that are randomly selected within the population and who do not have any discernable phenotype or disease.

As used herein, a “group of target DNA sequences” refers to the simultaneous testing of a test genomic DNA sample for the presence of at least two, preferably ten and more preferably twenty different target DNA sequences.

### BRIEF DESCRIPTION OF THE FIGURES

FIG. 1 shows a schematic representation of one embodiment of a method for determining differences in the genomic representation of an elected target genomic DNA sequence. T1 and T2 refer to different upstream and downstream tag sequences, respectively. T1′ and T2′ refer to the complement of tag sequences T1 and T2.

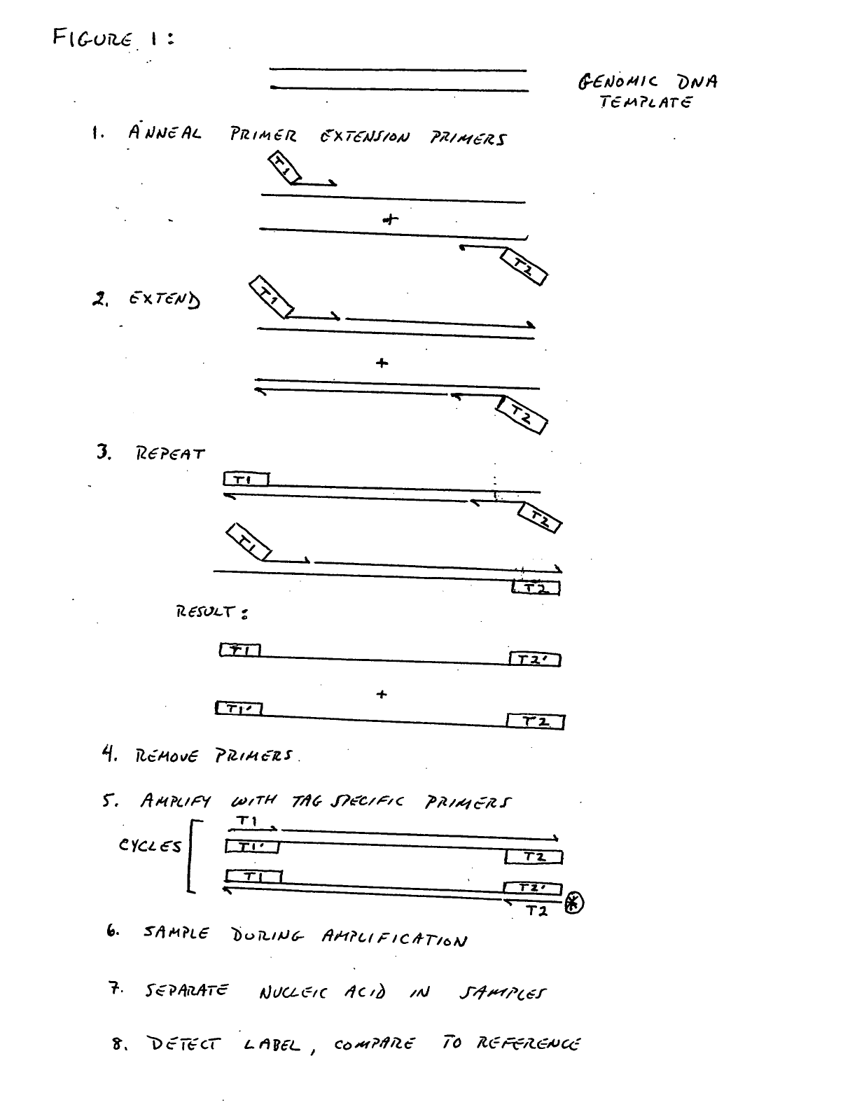

### DETAILED DESCRIPTION OF THE INVENTION

The invention provides methods of determining the variation in the representation of specific target DNA sequences within a genomic DNA sample. The methods of the invention employ primer extension reactions that incorporate sequence tags permitting the simultaneous identification of multiple specific target DNA sequences. Tagged fragments are then amplified using sets of primers specific for the tags wherein the downstream primer is labeled. During the amplification regimen, aliquots of the reaction are withdrawn and subjected to size separation and detection of the amplified fragments. The target DNA sequences are identified based on the size and identity of the label attached to the amplified fragments. Because both amplimer size and incorporated label are detected, the system is well suited for multiplexing. Further, the separation and detection can be performed during the amplification reaction, such that a profile of the amplification reaction is generated in real time. The real time aspect provides rapid analysis and comparison of the representation of the target sequence relative to a reference sample as well as information regarding the course of the amplification that is useful in identifying and eliminating artifactual signals caused, for example, by interactions between primers.

The practice of the present invention will employ, unless otherwise indicated, conventional techniques of molecular biology, microbiology and recombinant DNA techniques, which are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzmmology (Academic Press, Inc.); Short Protocols In Molecular Biology, (Ausubel et al., ed., 1995).

Primer Synthesis

Methods for synthesizing primers are available in the art. The oligonucleotide primers of this invention can be prepared using any conventional DNA synthesis method, such as, phosphotriester methods such as described by Narang et al. (1979, Meth. Enzymol., 68:90) or Itakura (U.S. Pat. No. 4,356,270), or and phosphodiester methods such as described by Brown et al. (1979, Meth. Enzymol., 68:109), or automated embodiments thereof, as described by Mullis et al. (U.S. Pat. No. 4,683,202). Also see particularly Sambrook et al.(1989), Molecular Cloning: A Laboratory Manual (2d ed.; Cold Spring Harbor Laboratory: Plainview, N.Y.), herein incorporated by reference.

Generating Sequence Tagged Primer Extension Products:

Interrogation of a Single DNA Target Sequence

As a first step, the invention involves the generation of sequence-tagged primer extension products. A critical aspect of this step is that the tag on any particular extension product specifically corresponds with the identity of an elected target DNA sequence. In this step, the tag is incorporated by the extension of a primer with the following general structure:

5′-Tag_{c}-target complement-3′

wherein “Tag_{c}” is the tag sequence that corresponds with the identity of the elected target DNA sequence at the 3′ terminus of the primer, and “target complement” is the 3′ region of the primer that specifically hybridizes to the elected target DNA sequence. The Tagc sequence is preferably 20 to 30 nucleotides in length and preferably does not hybridize under primer extension conditions to a sequence in the genome of the organism being genotyped or to any of the other primers used in a given reaction. Tag sequences as used throughout this specification are preferably high in GC content, thus enabling the use of high annealing temperatures in the subsequent PCR amplification step. This ensures a higher specificity of hybridization of the tag-specific primers to the Tag_{c} sequences and the avoidance of possible artefactual PCR products that tend to interfere with multiplex amplifications. The “target complement” is long enough to provide specific hybridization and will generally be about 10 to 25 nucleotides in length.

Sequence-tagged upstream primers are used to generate the opposite strand of a given target DNA sequence. These primers will have the general structure:

5′-Tag-target complement-3′

wherein “Tag” refers to a sequence tag different from each of those used in a downstream primer extension primer or downstream set of primer extension primers, and “target complement” refers to a sequence complementary to a region upstream of the elected DNA target sequence. The “Tag” sequence on the upstream primer is preferably 20 to 30 nucleotides in length and preferably does not hybridize under primer extension conditions to a sequence in the genome of the organism being genotyped, or to any of the other primers being used in a given reaction. In a preferred embodiment, the “Tag” will have the same or within 5-10% of the GC content of the “Tagc” sequence. The “target complement” is long enough to provide specific hybridization under primer extension conditions between the primer and a sequence upstream of an elected DNA target sequence, and will generally be about 10 to 25 nucleotides in length. The distance upstream will generally be at least 50 nucleotides, but can be 50 to 1000 nucleotides or more, preferably 50 to 500, or 50 to 250 nucleotides upstream of the elected DNA target sequence. The distance of the upstream primer sequence from the elected DNA target sequence determines the size or length of the later amplification products. The sizes of the later amplification products must be selected so as to differ by more than the resolution limit of the system used for size separation. Thus, if the limit of resolution of separation is one base, the sizes of the amplification products should be selected to differ by at least one base in length, and preferably more (e.g., at least 5, 10, 15 bases or more). When the limit of resolution is, for example, 10 bases, sizes of the amplification products should differ by at least 10 bases, and preferably more (e.g., at least 15, 20, 25, 30 bases or more). In a preferred embodiment, the “target complement” of the upstream primers will have the same or within 5-10% of the GC content of the “target complement” sequence of the downstream primers.

The terms “upstream” and “downstream” are used herein in order to facilitate the description of the invention. However, it is recognized that because of the double-stranded nature of DNA, an elected DNA target sequence could be approached with elected DNA target sequence-specific primers from either side, that is, from upstream or downstream, by hybridization of the primer to one strand as opposed to the other. The invention specifically contemplates the interrogation of target DNA sequences on either strand of the genomic DNA.

In order to generate sequence-tagged primer extension products according to the invention, a nucleic acid sample is denatured, preferably by heat, e.g., to 95° C. for 2 minutes or more, and allowed to re-anneal in the presence of an upstream extension primer and a set of downstream primer extension primers for each elected DNA target sequence to be interrogated in the reaction. The denaturing and annealing is best performed in a buffer compatible with the nucleic acid polymerase to be used for the primer extension reaction, e.g., 1× Taq polymerase buffer. Re-annealing is performed at a temperature below the Tₘ of the primers, generally between about 20° C. and 60° C., although lower or higher temperatures may be suitable for some primers. Primers should be present at about 15 to 500 nM for each primer. Optimal primer concentrations can be determined empirically by one of skill in the art with a minimum of experimentation, for example by setting up test reactions in which the primers are varied over the 15 to 500 nM range and analyzing the results with respect to the relative resolution, yield and specificity of the extension or amplification reactions.

Following annealing in the presence of the primers, polymerization is performed using a nucleic acid polymerase. Numerous polymerases sufficient for this step are known and can be selected by one skilled in the art (see section “DNA Polymerases useful according to the invention,” below). Among the most commonly used enzymes are the thermostable Taq polymerase and other thermostable polymerases, e.g., Pfu polymerase. Primer extension is performed under standard conditions for the enzyme chosen, e.g., 50 mM KCl, 10 mM Tric-HCl (pH 8.8@ 25° C.), 0.5 to 3 mM MgCl₂, and 0.1% BSA and 100 μM each dNTP at 72° C. for two minutes.

The first round of primer extension results in a population in which one strand has an upstream primer and tag sequence incorporated and the other strand has a downstream primer and tag sequence incorporated. The incorporation of the downstream primer necessarily incorporates the tag sequence associated with or corresponding to the elected DNA target sequence. In order to generate a population in which molecules representing each strand carry both an upstream tag or its complement and a downstream tag or its complement, the products of the first primer extension reaction are subjected to another round of denaturing, re-annealing in the presence of the same primers, and polymerase extension of those primers.

Following the second round of primer extension, non-extended primers are removed. Any method of primer removal can be used, e.g., electrophoresis or column chromatography, but it is preferred that a heat labile exonuclease specific for single-stranded DNA be used. The use of a heat-labile exonuclease avoids the need for time-consuming separation and purification procedures and the possibility for contamination or sample loss. Heat labile exonucleases useful according to the invention include, for example E. coli Exonuclease I (ExoI), and Exonuclease VII (ExoVII). Exol, for example, is active at 37° C. but is inactivated by incubation for 20 minutes at 80° C.

The primers used for primer extension are removed so that new primers, corresponding to the incorporated upstream and downstream tag sequences, can be used to amplify the primer extension products. Following the removal of the first primers, a set of primers comprising an upstream tag sequence primer and downstream tag sequence primers are added. Each downstream tag sequence primer is distinguishably labeled (e.g., end labeled) with a fluorescent dye. The mixture with the new primers added is then subjected to an amplification regimen comprising cycles of thermal denaturation, re-annealing and polymerase extension. The amplification regimen should comprise at least two cycles, but will preferably comprise 2 to 35 cycles, more preferably 10 to 30 cycles, and more preferably 15 to 25 cycles.

During the cycling regimen, following at least one of the cycles of denaturation, primer annealing and primer extension in this aspect of the invention, a sample or aliquot of the reaction is withdrawn from the tube or reaction vessel, and nucleic acids in the aliquot are separated and detected. The separation and detection are performed concurrently with the cycling regimen, such that a curve representing product abundance as a function of cycle number can be generated while the cycling occurs. As used herein, the term “concurrently” means that the separation is at least initiated while the cycling regimen is proceeding. Depending upon the separation technology used (e.g., capillary electrophoresis) and the number and size of species to be separated in a given reaction, the separation will most often require on the order of 1-120 minutes per aliquot. Thus, when separation steps take longer than the duration of each cycle, and when samples are withdrawn after, for example, every cycle, the separation steps will be completed after the completion of the full cycling regimen. However, as used herein, this situation is still considered to be “concurrent” separation, as long as the separation of each sample was initiated during the cycling regimen. Concurrent separation is most preferably performed through use of a robotic sampler that deposits the samples to the separation apparatus immediately after the samples are withdrawn from the cycling reaction.

In the manner described above, the presence and relative representation of an elected DNA target sequence is determined by detection of the fluorescent signals on the size-separated amplification products in comparison with a reference. Because the tag on a given primer corresponds to the identity of the elected DNA target sequence of the original downstream primer extension primer, the incorporation and detection of that fluorescently labeled tag identifies the elected DNA target sequence.

Interrogation of a Group of DNA Target Sequences

In a preferred aspect, the original primer extension reactions include primer sets specific for more than one elected DNA target sequence. In this aspect, each different DNA target sequence will be represented by a distinctly sized amplification product. For example, one can include additional upstream primers, each comprising the same tag sequence and varying in the 3′ region that hybridizes at a distinct distance upstream of an additional known DNA target sequence. Following two rounds of primer extension and the removal of non-incorporated primers as described above, a single amplification primer set is used, identical to that used when a single elected DNA target sequence is interrogated. That is, the amplification primer set will comprise an upstream primer containing the upstream tag and a set of distinguishably labeled downstream primers comprising the tags on the downstream primer extension primers, where the labels correspond to the downstream tags which in turn correspond to the sequence of elected DNA target sequences. Each elected DNA target sequence interrogated will have a distinct size when separated, and the identity of the label incorporated into a molecule of that size positively identifies the elected DNA target sequence. The ability to amplify and detect multiple elected DNA target sequences with a single set of amplification primers has the advantage of avoiding primer interaction problems prevalent when large numbers of primers are used for amplification. In addition, the effect of variations in primer annealing efficiency will be largely negated because all elected DNA target sequences interrogated with a given amplification primer set will be affected by such variations to the same degree.

Further multiplexing can be achieved by using more than one set of upstream and downstream primer extension tag sequences. The additional sets will comprise tags distinct from those used in other sets. Care should be taken to avoid tags with complementarity to other tags to be used simultaneously. As above, each set will comprise upstream tags selected so that the ultimate amplification products will be distinctly sized, and downstream tags in which the respective tags correspond to each elected DNA target sequence of the primer extension primers. For the amplification step, the downstream primers can be labeled with the same corresponding fluorescent labels as the other sets, or, preferably with a different set of distinguishable fluorescent labels. Following size separation, the amplified DNA target sequence-containing fragments are identified by size, and the identity of the elected DNA target sequence is identified by the label incorporated, as described above.

In a preferred embodiment, the invention provides for downstream primer extension primers in which the target complement is a repetitive DNA element.

In a preferred embodiment, the invention provides for downstream primer extension primers in which the target complement is a rearranged DNA element, for example a transposable DNA element.

In a preferred embodiment, the invention provides for downstream primer extension primers in which the target complement is an arbitrary DNA sequence.

In another preferred embodiment, the invention provides for the batch wise processing of at least two test genomic DNA samples, preferably ten test genomic DNA samples, preferably fifty test genomic DNA samples, preferably 500 test genomic DNA samples or more preferably 1000 test genomic DNA samples.

General Considerations for Primer Design

Oligonucleotide primers are generally 5 to 100 nucleotides in length, preferably from 17 to 45 nucleotides, although primers of different lengths are of use. Primers for primer extension reactions are preferably 10 to 60 nucleotides long, while primers for amplification are preferably about 17-25 nucleotides in length. Primers useful according to the invention can be designed to have a particular melting temperature (Tₘ) by the method of melting temperature estimation. Commercial programs, including Oligo™, Primer Design and programs available on the internet, including Primer3 and Oligo Calculator can be used to calculate the Tₘ of a polynucleotide sequence useful according to the invention. Preferably, the Tₘ of an amplification primer useful according to the invention (e.g., a tag sequence), as calculated for example by Oligo Calculator, is between about 45° C. and 65° C. and more preferably between about 50° C. and 60° C.

The Tₘ of a polynucleotide affects its hybridization to another polynucleotide (e.g., the annealing of an oligonucleotide primer to a template polynucleotide). In the methods of the invention, it is preferred that the oligonucleotide primers used in various steps selectively hybridize to a target template or to polynucleotides derived from the target template. Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., 1984, Polynucleotide Res. 12:203, incorporated herein by reference. As a result, it is expected that a certain degree of mismatch at the priming site is tolerated. Such mismatch may be small, such as a mono-, di- or tri-nucleotides. Alternatively, a region of mismatch may encompass loops, which are defined as regions in which there exists a mismatch in an uninterrupted series of four or more nucleotides.

Numerous factors influence the efficiency and selectivity of hybridization of the primer to a second polynucleotide molecule. These factors, which include primer length, nucleotide sequence and/or composition, hybridization temperature, buffer composition and potential for steric hindrance in the region to which the primer is required to hybridize, will be considered when designing oligonucleotide primers according to the invention.

A positive correlation exists between primer length and both the efficiency and accuracy with which a primer will anneal to a target sequence. In particular, longer sequences have a higher melting temperature (T_{M}) than do shorter ones, and are less likely to be repeated within a given target sequence, thereby minimizing promiscuous hybridization. Primer sequences with a high G-C content or that comprise palindromic sequences tend to self-hybridize, as do their intended target sites, since unimolecular, rather than bimolecular, hybridization kinetics are generally favored in solution. However, it is also important to design a primer that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with primer annealing efficiency, as does the concentration of organic solvents, e.g. formamide, that might be included in a priming reaction or hybridization mixture, while increases in salt concentration facilitate binding. Under stringent annealing conditions, longer hybridization probes or synthesis primers hybridize more efficiently than do shorter ones, which are sufficient under more permissive conditions. Preferably, stringent hybridization is performed in a suitable buffer (for example, 1× Taq Polymerase Buffer, or other buffer suitable for enzymes used for primer extension and amplification) under conditions that allow the polynucleotide sequence to hybridize to the oligonucleotide primers. Stringent hybridization conditions can vary (for example from salt concentrations of less than about 1M, more usually less than about 500 mM and preferably less than about 200 mM) and hybridization temperatures can vary (for example, from as low as 0° C. to greater than 22° C., greater than about 30° C., and (most often) in excess of about 37° C.) depending upon the lengths and/or the polynucleotide composition or the oligonucleotide primers. Longer fragments may require higher hybridization temperatures for specific hybridization. As several factors affect the stringency of hybridization, the combination of parameters is more important than the absolute measure of a single factor.

Unlike the design of primers made to recognize a sequence anywhere on a given gene, primers designed to hybridize near a known elected DNA target sequence are limited with respect to the modifications one can make to manipulate Tₘ. For example, where one would normally be able to shift up- or downstream on a sequence to find a region with a more favorable GC content, when a primer is designed to hybridize adjacent to an elected DNA target sequence, one cannot move the primer to another location. In this situation, then, the primary means of manipulating Tₘ is to vary the length of the complementary sequence in the primer.

Sequence Tags Useful According to the Invention

Tags useful according to the invention are preferably heterologous or artificial nucleotide sequences of at least 15, and preferably 20 to 30 nucleotides in length. A tag will preferably not hybridize under PCR annealing conditions to a sequence in the genome of the organism being genotyped. A tag sequence according to the invention can be, but is not necessarily arbitrary. One can determine whether a potential tag sequence hybridizes under PCR annealing conditions to a sequence in the genome of an organism by using the tag sequence as a labeled primer in a primer extension reaction with genomic DNA from the organism of interest as template. The labeled primer is annealed to the genomic DNA at the annealing temperature one plans to use for the amplification steps of the method of the invention, and then incubated with thermostable polymerase under extension conditions. The reaction products are then electrophoretically separated alongside labeled probe alone. If the labeled tag appears in a band or bands larger than the tag primer, the tag primer hybridized under PCR annealing conditions to a sequence in the genome of the organism being genotyped. Care should also be taken to avoid tags with complementarity to other tags intended for use in the same reaction.

DNA Polymerases Useful According to the Invention

DNA polymerases and their properties are described in detail in, among other places, DNA Replication 2nd edition, Kornberg and Baker, W. H. Freeman, New York, N.Y. (1991).

Known conventional DNA polymerases include, for example, Pyrococcus furiosus (Pfu) DNA polymerase (Lundberg et al., 1991, Gene, 108:1, provided by Stratagene), Pyrococcus woesei (Pwo) DNA polymerase (Hinnisdaels et al., 1996, Biotechniques, 20:186-8, provided by Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent DNA polymerase, Cariello et al., 1991, Polynucleotides Res, 19: 4193, provided by New England Biolabs), 9°Nm DNA polymerase (discontinued product from New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127:1550), Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep-Vent DNA polymerase, Juncosa-Ginesta et al., 1994, Biotechniques, 16:820, provided by New England Biolabs), UlTma DNA polymerase (from thermophile Thermotoga maritima; Diaz and Sabino, 1998 Braz J. Med. Res, 31:1239; provided by PE Applied Biosystems), Tgo DNA polymerase (from thermococcus gorgonarius, provided by Roche Molecular Biochemicals), E. coli DNA polymerase I (Lecomte and Doubleday, 1983, Polynucleotides Res. 11:7505), T7 DNA polymerase (Nordstrom et al., 1981, J. Biol. Chem. 256:3112), and archaeal DP1/DP2 DNA polymerase II (Cann et al., 1998, Proc Natl Acad Sci USA 95:14250-5). The polymerization activity of any of the above enzymes can be defined by means well known in the art. One unit of DNA polymerization activity of conventional DNA polymerase, according to the subject invention, is defined as the amount of enzyme which catalyzes the incorporation of 10 nmoles of total deoxynucleotides (dNTPs) into polymeric form in 30 minutes at optimal temperature (e.g., 72° C. for Pfu DNA polymerase). Assays for DNA polymerase activity and 3′-5′ exonuclease activity can be found in DNA Replication 2nd Ed., Kornberg and Baker, supra; Enzymes, Dixon and Webb, Academic Press, San Diego, Calif. (1979), as well as other publications available to the person of ordinary skill in the art.

Labeling of Oligonucleotide Primers

Oligonucleotide primers useful according to the invention can be labeled, as described below, by incorporating moieties detectable by spectroscopic, photochemical, biochemical, immunochemical, enzymatic or chemical means. The method of linking or conjugating the label to the oligonucleotide primer depends, of course, on the type of label(s) used and the position of the label on the primer (i.e., 3′-terminal, 5′-terminal or body-labeled).

While fluorescent dyes are preferred, a variety of labels that would be appropriate for use in the invention, as well as methods for their inclusion in the primer, are known in the art and include, but are not limited to, enzymes (e.g., alkaline phosphatase and horseradish peroxidase) and enzyme substrates, radioactive atoms, chromophores, fluorescence quenchers, chemiluminescent labels, and electrochemiluminescent labels, such as Origen™ (Igen), that may interact with each other to enhance, alter, or diminish a signal. Of course, if a labeled molecule is used in a PCR based amplification assay involving thermal cycling, the label must be able to survive the temperature cycling required in this automated process. Ideally, four distinguishable labels that can be detected using similar equipment, methods and/or substrates are preferred.

Fluorophores for use as labels in constructing labeled primers of the invention include, but are not limited to rhodamine and derivatives (such as Texas Red), fluorescein and derivatives (such as 5-bromomethyl fluorescein), Cy5, Cy3, JOE, FAM, Oregon Green™, Lucifer Yellow, IAEDANS, 7-Me₂N-coumarin-4-acetate, 7-OH-4-CH₃-coumarin-3-acetate, 7-NH₂-4-CH₃-coumarin-3-acetate (AMCA), monobromobimane, pyrene trisulfonates, such as Cascade Blue, and monobromorimethyl-ammoniobimane. In general, fluorophores with wide Stokes shifts are preferred, to allow using fluorimeters with filters rather than a monochromometer and to increase the efficiency of detection.

The labels can be attached to the oligonucleotide directly or indirectly by a variety of techniques. Depending on the precise type of label or tag used, the label can be located at the 5′ end of the primer or located internally in the primer, or attached to spacer arms of various sizes and compositions to facilitate signal interactions. 5′ end labeling is preferred. Using commercially available phosphoramidite reagents, one can produce oligomers containing functional groups (e.g., thiols or primary amines) at the 5′-terminus via an appropriately protected phosphoramidite, and can label them using protocols described in, for example, PCR Protocols: A Guide to Methods and Applications, Innis et al., eds. Academic Press, Ind., 1990.

Methods for introducing oligonucleotide functionalizing reagents to introduce one or more sulfhydryl, amino or hydroxyl moieties into the oligonucleotide primer sequence, typically at the 5′ terminus, are described in U.S. Pat. No. 4,914,210. A 5′ phosphate group can be introduced as a radioisotope by using polynucleotide kinase and gamma-³²P-ATP or gamma-³³P-ATP to provide a reporter group. Biotin can be added to the 5′ end by reacting an aminothymidine residue, or a 6-amino hexyl residue, introduced during synthesis, with an N-hydroxysuccinimide ester of biotin.

Amplification

PCR methods are well-known to those skilled in the art, such as those described in Mullis and Faloona, 1987, Methods Enzymol., 155: 335, Saiki et al., 1985, Science 230:1350, and U.S. Pat. Nos. 4,683,202, 4,683,195 and 4,800,159, each of which is incorporated herein by reference. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment whose termini are defined by the 5′ ends of the primers. PCR is reported to be capable of producing a selective enrichment of a specific DNA sequence by a factor of 10⁹.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of skill in the art. An annealing temperature between 20° C. and 72° C. is most commonly used. Initial denaturation of the template molecules is normally achieved by incubation at 92° C. to 99° C. for 4 minutes, followed by 20-40 cycles consisting of denaturation (94° C. for 15 seconds to 1 minute), annealing (temperature based on Tₘ as discussed above, usually about 5° C. below the Tₘ of the oligonucleotide in the reaction with the lowest Tₘ; usually 1-2 minutes), and extension (usually 72° C. for 1-3 minutes).

Sampling

Sampling during the amplification regimen can be performed at any frequency or in any pattern desired. It is preferred that sampling occurs after each cycle in the regimen, although less frequent sampling can also be used, for example, every other cycle, every third cycle, every fourth cycle, etc. While a uniform sample interval will most often be desired, there is no requirement that sampling be performed at uniform intervals. As just one example, the sampling routine may involve sampling after every cycle for the first five cycles, and then sampling after every other cycle.

Sampling can be as simple as manually pipetting an aliquot from the reaction, but is preferably automated such that the aliquot is automatically withdrawn at predetermined sampling intervals. It is preferred that the reaction mixture is replenished at each withdrawal with equal volumes of fresh components such as dNTPs, primers and DNA polymerase. For this and other aspects of the invention, it is preferred, although not necessary that the cycling be performed in a microtiter or multiwell plate format. This format, which uses plates comprising multiple reaction wells, not only increases the throughput of the assay process, but is also well adapted for automated sampling steps due to the modular nature of the plates and the uniform grid layout of the wells on the plates. Common microtiter plate designs useful according to the invention have, for example 12, 24, 48, 96, 384 or more wells, although any number of wells that physically fit on the plate and accommodate the desired reaction volume (usually 10-100 μl) can be used according to the invention. Generally, the 96 or 384 well plate format is preferred.

An automated sampling process can be readily executed as a programmed routine and avoids both human error in sampling (i.e., error in sample size and tracking of sample identity) and the possibility of contamination from the person sampling. Robotic samplers capable of withdrawing aliquots from thermal cyclers are available in the art. For example, the Mitsubishi RV-E2 Robotic Arm can be used in conjunction with a SciClone™ Liquid Handler or a Robbins Scientific Hydra 96 pipettor.

The robotic sampler useful according to the invention can be integrated with the thermal cycler, or the sampler and cycler can be modular in design. When the cycler and sampler are integrated, thermal cycling and sampling occur in the same location, with samples being withdrawn at programmed intervals by a robotic sampler. When the cycler and sampler are modular in design, the cycler and sampler are separate modules. In one embodiment, the assay plate is physically moved, e.g., by a robotic arm, from the cycler to the sampler and back to the cycler.

The volume of an aliquot removed at the sampling step can vary, depending, for example, upon the total volume of the amplification reaction, the sensitivity of product detection, and the type of separation used. Amplification volumes can vary from several microliters to several hundred microliters (e.g., 5 μl, 10 μl, 20 μl, 40 μl, 60 μl, 80 μl, 100 μl, 120 μl, 150 μl, or 200 μl or more), preferably in the range of 10-150 μl, more preferably in the range of 10-100 μl. Aliquot volumes can vary from 0.1 to 30% of the reaction mixture.

Separation of Nucleic Acids

Separation of nucleic acids according to the invention can be achieved by any means suitable for separation of nucleic acids, including, for example, electrophoresis, HPLC or mass spectrometry. Due to its speed and resolution, separation is preferably performed by capillary electrophoresis (CE).

CE is an efficient analytical separation technique for the analysis of minute amounts of sample. CE separations are performed in a narrow diameter capillary tube, which is filled with an electrically conductive medium termed the “carrier electrolyte.” An electric field is applied between the two ends of the capillary tube, and species in the sample move from one electrode toward the other electrode at a rate which is dependent on the electrophoretic mobility of each species, as well as on the rate of fluid movement in the tube. CE may be performed using gels or liquids, such as buffers, in the capillary. In one liquid mode, known as “free zone electrophoresis,” separations are based on differences in the free solution mobility of sample species. In another liquid mode, micelles are used to effect separations based on differences in hydrophobicity. This is known as Micellar Electrokinetic Capillary Chromatography (MECC).

CE separates nucleic acid molecules on the basis of charge, which effectively results in their separation by size or number of nucleotides. When a number of fragments are produced, they will pass the fluorescence detector near the end of the capillary in ascending order of size. That is, smaller fragments will migrate ahead of larger ones and be detected first.

CE offers significant advantages of over conventional electrophoresis, primarily in the speed of separation, small size of the required sample (on the order of 1-50 nl), and high resolution. For example, separation speeds using CE can be 10 to 20 times faster than conventional gel electrophoresis, and no post-run staining is necessary. CE provides high resolution, separating molecules in the range of about 10-1,000 base pairs differing by as little as a single base pair. High resolution is possible in part because the large surface area of the capillary efficiently dissipates heat, permitting the use of high voltages. In addition, band broadening is minimized due to the narrow inner diameter of the capillary. In free-zone electrophoresis, the phenomenon of electroosmosis, or electroosmotic flow (EOF) occurs. This is a bulk flow of liquid that affects all of the sample molecules regardless of charge. Under certain conditions EOF can contribute to improved resolution and separation speed in free-zone CE.

CE can be performed by methods well known in the art, for example, as disclosed in U.S. Pat. Nos. 6,217,731; 6,001,230; and 5,963,456, which are incorporated herein by reference. High throughput CE equipment is available commercially, for example, the HTS9610 High Throughput Analysis System and SCE 9610 fully automated 96-capillary electrophoresis genetic analysis system from Spectrumedix Corporation (State College, Pa.). Others include the P/ACE 5000 series from Beckman Instruments Inc (Fullerton, Calif.) and the ABI PRISM 3100 genetic analyzer (Applied Biosystems, Foster City, Calif.). Each of these devices comprises a fluorescence detector that monitors the emission of light by molecules in the sample near the end of the CE column. The standard fluorescence detectors can distinguish numerous different wavelengths of fluorescence emission, providing the ability to detect multiple fluorescently labeled species in a single CE run from an amplification sample.

Another means of increasing the throughput of the CE separation is to use a plurality of capillaries, or preferably an array of capillaries. Capillary Array Electrophoresis (CAE) devices have been developed with 96 capillary capacity (e.g., the MegaBACE instrument from Molecular Dynamics) and higher, up to and including even 1000 capillaries. In order to avoid problems with the detection of fluorescence from DNA caused by light scattering between the closely juxtaposed multiple capillaries, a confocal fluorescence scanner can be used (Quesada et al., 1991, Biotechniques 10:616-25).

The apparatus for separation (and detection) can be separate from or integrated with the apparatus used for thermal cycling and sampling. Because according to the invention the separation step is initiated concurrently with the cycling regimen, samples are preferably taken directly from the amplification reaction and placed into the separation apparatus so that separation proceeds concurrently with amplification. Thus, while it is not necessary, it is preferred that the separation apparatus is integral with the thermal cycling and sampling apparatus. In one embodiment, this apparatus is modular, comprising a thermal cycling module and a separation/detection module, with a robotic sampler that withdraws sample from the thermal cycling reaction and places it into the separation/detection apparatus.

Detection

Amplification product detection methods useful according to the invention measure the intensity of fluorescence emitted by labeled primers when they are irradiated with light within the excitation spectrum of the fluorescent label. Fluorescence detection technology is highly developed and very sensitive, with documented detection down to a single molecule in some instances. High sensitivity fluorescence detection is a standard aspect of most commercially-available plate readers, microarray detection set-ups and CE apparatuses. For CE equipment, fiber optic transmission of excitation and emission signals is often employed. Spectrumedix, Applied Biosystems, Beckman Coulter and Agilent each sell CE equipment with fluorescence detectors sufficient for the fluorescence detection necessary for the methods described herein.

The fluorescence signals from two or more different fluorescent labels can be distinguished from each other if the peak wavelengths of emission are each separated by 20 nm or more in the spectrum. Generally the practitioner will select fluorophores with greater separation between peak wavelengths, particularly where the selected fluorophores have broad emission wavelength peaks. It follows that the more different fluorophores one wishes to include and detect concurrently in a sample, the narrower should be their emission peaks.

Sequencing

The differentially represented amplified products can be purified and sequenced to verify or determine the identity of the amplified DNA fragment. Amplified products may be purified to get rid of free primers used in the amplification by methods known in the art (e.g., Current Protocols in Molecular Biology, supra). In a preferred embodiment, the PCR products are purified using the High Pure PCR Product Purification Kit (Roche, Cat #1-732-676).

Any known sequencing method known in the art can be used to sequence the PCR products. In a preferred embodiment, the sequencing is carried out by using one of the PCR amplification primers.

Diseases and Disease-Related Genes According to the Invention

In many cases, disease or predisposition to disease is the result of one or more genetic factors. The methods disclosed in the present invention are useful as a diagnostic tool to screen for known genetic alterations or to identify and characterize new genetic alterations that are associated with a particular disease. In many instances, for example as with various forms of cancer, disease onset is often insidious and disease progression occurs as a result of a steady accrual of mutations in genes that are often involved in DNA repair mechanisms. Thus genetic screening assays, as disclosed in the present invention, promise to provide the physician with tools that permit the early diagnosis of a susceptibility to a disease before a clear manifestation of the disease is evident.

Diseases or disease susceptibilities that can be screened for genetic alterations using the methods of the invention include any disease or disorder known or suspected to be associated with a differential genomic sequence representation. For example, diseases or disease susceptibilities include, but are not limited to, hypertension, blood lipid diseases, cancer and cancer susceptibility, diabetes, manic depression, anxiety, schizophrenia, Gaucher disease, cystic fibrosis and sickle cell anemia, blood lipid diseases, cancer susceptibility, acne, baldness and sleep disorders. The GeneCards WWW database of disease-related genes (on the World Wide Web at nciarray.nci.nih.gov/cards) contains an extensive list of known disease or disease predisposing genes and information on their loci and GenBank accession numbers for those genes that have been cloned. Representative, but non-limiting, examples of diseases according to the invention are listed below.

Hypertension

Multiple genes cause or are very closely linked to hypertension or a predisposition to it. These include: AGTR1, which encodes angiotensin receptor 1 (GenBank Accession No. S77410); AGT, which encodes angiotensinogen (GenBank Accession No. K02215); GNB3, which encodes guanine nucleotide binding protein (G protein) β polypeptide 3 (GenBank Accession No. U47924); HSD11B2, which encodes hydroxysteroid (11-β) dehydrogenase 2 (GenBank Accession No. U26726); NPR3, which encodes natriuretic peptide receptor C/guanylate cyclase C (GenBank Accession No. M59305); PNMT, which encodes phenylethanolamine N-methyltransferase (GenBank Accession No. X52730); PPH1, which encodes primary pulmonary hypertension 1 (localized to 2q31-q32); and SAH, which encodes the human homolog of the rat SA hypertension-associated gene (localized to 16p13.11). The preceding list is not intended to be limiting. Any of these genes may have polymorphisms or other mutation which are useful in predicting disease occurrence or susceptibility, or which affect the response of an individual to antihypertensive drugs. The genetic basis of hypertension is further described below.

Lifton reviewed the molecular genetics of human blood pressure variation. He pointed out that at least 10 genes have been shown to alter blood pressure; most of these are rare mutations imparting large quantitative effects that either raise or lower blood pressure (Lifton, 1996, Science 272: 676). These mutations alter blood pressure through a common pathway, changing salt and water reabsorption in the kidney. Disorders that fall into this category include glucocorticoid remediable aldosteronism, the syndrome of apparent mineralocorticoid excess, and Liddle syndrome, which is known to be caused by a mutation in either the beta subunit or the gamma subunit of the renal epithelial sodium channel.

Several varieties of familial, salt-sensitive, low-renin hypertension with a proven or presumptive genetic basis have been described (Gordon, 1995, Nature Genet. 11: 6). The conditions in which the molecular basis of the disorder has been identified at the DNA level include 2 forms of Liddle syndrome due to mutation in the beta subunit or gamma subunit of the amiloride-sensitive epithelial sodium channel; the syndrome of apparent mineralocorticoid excess (AME) due to a defect in the renal form of 11-beta-hydroxysteroid dehydrogenase; and the form of familial hyperaldosteronism which is successfully treated with low doses of glucocorticoids, such as dexamethasone (“glucocorticoid-remediable aldosteronism”), which is due to a Lapore hemoglobin-like fusion of the contiguous CYP11B1 and CYP11B2 genes.

Diabetes

There are several forms of diabetic disease. These include Insulin Dependent Diabetes Mellitus (IDDM), Non-Insulin Dependent Diabetes Mellitus (NIDDM), Maturity Onset Diabetes in the Young (MODY, a subtype of NIDDM), and Diabetes Insipidus. The diseases, evidence for genetic influences in the diseases and drugs used to treat them are discussed below.

Insulin Dependent Diabetes Mellitus (Type I Diabetes):

The type of diabetes mellitus called IDDM is a disorder of glucose homeostasis that is characterized by susceptibility to ketoacidosis in the absence of insulin therapy. It is a genetically heterogeneous autoimmune disease affecting about 0.3% of Caucasian populations (Todd, 1990). Genetic studies of IDDM have focused on the identification of loci associated with increased susceptibility to this multifactorial phenotype.

Currently, diabetes mellitus is classified clinically into 2 major forms of the primary illness, insulin-dependent diabetes mellitus (IDDM) and noninsulin-dependent diabetes mellitus (NIDDM), and secondary forms related to gestation or medical disorders. Appearance of the IDDM phenotype is thought to require a predisposing genetic background and interaction with other environmental factors. Rotter and Rimoin (1978) hypothesized that there are at least 2 forms of IDDM: a form characterized by pancreatic autoimmunity, and a form characterized by antibody response to exogenous insulin. Interestingly, the DR3 and DR4 alleles associated with these forms, respectively, seem to have a synergistic effect on the predisposition to IDDM based on the greatly increased risk observed in persons having both alleles the B8 and B15 antigens (Svejgaard and Ryder, 1977). IDDM exhibits 30-50% concordance in monozygotic twins suggesting that the disorder is dependent on environmental factors as well as genes.

An example of an autosomal dominant type of diabetes mellitus is maturity-onset diabetes of the young (MODY, see below). However, this mode of inheritance is not the case for other forms of diabetes.

Bell et al., (1984) described an association between IDDM and a polymorphic region in the 5-prime flanking region of the insulin gene (INS; 176730). This polymorphism (Bell et al., 1981) arises from a variable number of tandemly repeated (VNTR) 14-bp oligonucleotides. When divided into 3 size classes, a significant association was seen between the short-length (class I) alleles and IDDM. Several disease-associated polymorphisms were identified and the boundaries of association were mapped to a region of 19 kb on 11p15.5. Donald et al. (1989) used DR and DQ restriction fragment length polymorphisms (RFLPs) for linkage analysis and demonstrated very close linkage of an IDDM-susceptibility locus.

Lucassen et al. (1993) presented a detailed sequence comparison of the predominant haplotypes found in the region of 19 kb on 11p15.5 in a population of French-Canadian IDDM patients and controls. Identification of polymorphisms, both associated and unassociated with IDDM, permitted a further definition of the region of association to 4.1 kb. Ten polymorphisms within this region were found to be in strong linkage disequilibrium with each other and extended across the insulin gene locus and the VNTR situated immediately 5-prime to the insulin gene. These represent a set of candidate disease polymorphisms, one or more of which may account for the susceptibility to IDDM.

Davies et al. (1994) searched the human genome for genes that predispose to type 1 (insulin-dependent) diabetes mellitus. A total of 18 different chromosomal regions showed some positive evidence of linkage to the disease, strongly suggesting that IDDM is inherited in a polygenic fashion. Although the authors determined that no genes are likely to have as large effects as IDDM1 (in the major histocompatibility complex on 6p21), significant linkage was confirmed in the insulin gene region on 11p15 and established to 11q, 6q, and possibly to chromosome 18. Possible candidate genes within regions of linkage include GAD1 and GAD2, which encode the enzyme glutamic acid decarboxylase; SOD2, which encodes superoxide dismutase; and the Kidd blood group locus. Linkage of IDDM susceptibility to the region of the FGF gene on chromosome 11q13 was also reported by Hashimoto et al. (1994).

As discussed above, there are a number of genes for which genetic evidence suggest a role in diabetic disease or predisposition to it. These include: AQP2, encoding aquaporin 2 (diabetes insipidus; van Lieburg et al., 1994, Am. J. Hum. Genet. 55: 648-652); AVPR2, encoding arginine vasopressin receptor 2 (diabetes insipidus; Arthus, et al., 2000, J. Am. Soc. Nephrol. 11: 1044-1054); AVP, arginine vasopressin (diabetes insipidus; Rutishauser et al., 1996, J. Clin. Endocrinol. Metab. 81:192-198); GCGR, encoding glucagon receptor (Type II diabetes, Genbank Accession No. L20316); GCK, encoding glucokinase (MODY,Type 2; Burke et al., 1999, Biochem. J. 342: 345-352); GPD2, encoding glycerol-3-phosphate dehydrogenase 2 (Type II diabetes; Novials et al., 1997, Biochem. Biophys. Res. Comm. 231: 570-572); GYSI, encoding glycogen synthase 1 (susceptibility to Type II diabetes; Groop et al., 1993, New Eng. J. Med. 328: 10-14); HNF4A, encoding hepatocyte nuclear factor 4, alpha (MODY, type 1; Type II diabetes; Byrne et al., 1995, Diabetes 44: 699-704); IDDM10, encoding insulin-dependent diabetes mellitus 10 (Type I; Davies et al., 1994, Nature 371: 130-136; Reed et al., 1997, Hum. Molec. Genet. 6: 1011-1016); IDDM11, encoding insulin-dependent diabetes mellitus 11 (Type I; Corder et al., 2001, Ann. Hum. Genet. 65: 387-394); IDDM13, encoding insulin-dependent diabetes mellitus 13 (Type I; Fox et al., 2000, Am. J. Hum. Genet. 67: 67-81); IDDM15, encoding insulin-dependent diabetes mellitus 15 (Type I; European Consortium for IDDM Genome Studies, 2001, Am. J. Hum. Genet. 69: 1301-1313); IDDM2, encoding insulin-dependent diabetes mellitus 2 (Type I; Bennett et al., 1995, Nature Genet. 9: 284-292); IDDM3, encoding insulin-dependent diabetes mellitus 3 (Type I; Luo et al., 1995, Am. J. Hum. Genet. 57: 911-919); IDDM4, encoding insulin-dependent diabetes mellitus 4 (Type I; Eckenrode et al., 2000, Hum. Genet. 106: 14-18); IDDM5, encoding insulin-dependent diabetes mellitus 5 (Type I; Fox et al., 2000, Am. J. Hum. Genet. 67: 67-81); IDDM6, encoding insulin-dependent diabetes mellitus 6 (Type I; Merriman et al., 1997, Hum. Molec. Genet. 6: 1003-1010); IDDM7, encoding insulin-dependent diabetes mellitus 7 (Type I; Copeman et al., 1995, Nature Genet. 9: 80-85); IDDM8, encoding insulin-dependent diabetes mellitus 8 (Type I; Luo et al., 1995, Am. J. Hum. Genet. 57: 911-919); INSR, encoding insulin receptor (Type II diabetes; Hart et al., 1999, J. Clin. Endocr. Metab. 84: 1002-1006); INS, encoding insulin (Type I, one form of MODY; Abney et al., 2002, Am. J. Hum. Genet. 70: 920-934); IRS1, encoding insulin receptor substrate 1 (Type II diabetes; Almind et al., 1993, Lancet 342: 828-832); MODY1, encoding maturity onset diabetes of the young 1 (MODY, type 1; Type II diabetes; Bell et al., 1991, Proc. Nat. Acad. Sci. 88: 1484-1488); MODY3, encoding maturity onset diabetes of the young 3 (MODY, type 3; Type II diabetes; Ellard, 2000, Hum. Mutat. 16: 377-385); NIDDM1, encoding non-insulin-dependent diabetes 1 (Type II diabetes; Altshuler et al., 2000, Nature Genet. 26: 135-137); SLC2A2, encoding solute carrier family 2, member 2 (Type II diabetes; Akagi et al., 2000, J. Hum. Genet. 45: 60-62); SLC2A4, encoding solute carrier family 2, member 4 (Type II diabetes; Bell et al., 1989, Diabetes 38: 1072-1075); TCF1, encoding transcription factor 1 (HNF1; Chiu et al., 2000, J. Clin. Endocr. Metab. 85: 2178-2183), (MODY, type 3; Type II diabetes); and VP, encoding variegate porphyria (diabetes insipidus; Repaske et al., 1990, J. Clin. Endocr. Metab. 70: 752-757).

Manic Depression

Depressive disorders represent a prevalent (1 to 2%) and major illness characterized by episodes of dysphoria that are associated with somatic symptoms. It may have a manic-depressive (bipolar) or purely depressive (unipolar) course. The role of genetic factors is indicated by concordance in monozygotic and dizygotic twins, respectively, of 57% and 14%, and the correlation between adopted persons and their biologic relatives (Cadoret, 1978). The most characteristic features of bipolar affective disorder are episodes of mania (bipolar I, BP-I) or hypomania (bipolar II, BP-II) interspersed with periods of depression (Goodwin and Jamison, 1990). If untreated, manic-depressive illness is associated with a suicide rate of approximately 20%.

While the genetic basis for manic depressive illness is undeniable, the identification of the genes involved has been elusive. Linkage studies performed using polymorphic markers on a number of different families or populations exhibiting high incidence of the disease have shown possible linkages to certain HLA genotypes (Smeraldi et al., 1978; Weitkamp et al., 1981) and markers mapping to the 4p (Blackwood et al., 1996), 5q (Coon et al., 1993), 6p, 11 (Egeland et al., 1987), 9q34 (Sherrington et al., 1994), 18p (Berrettini et al., 1994), 18q22-23 (Stine et al., 1995), 21q22.3 (Straub et al., 1994) and Xq28 (Risch and Botstein, 1996) chromosomal loci. Genes thought to be involved in the disease or susceptibility to the disease include those encoding beta adrenoreceptor (Genbank Accession No. AFO22956) (Wright et al., 1984), tyrosine hydroxylase (Genbank Accession No.s D00292, D00291, D00290, D00289, D00288, D00287, D00286, D00285, D00284, D00283, D00282, D00281, D00280, D00279, D00278, D00277, D00276, D00275, D00274, D00273, D00272, D00271, D00270, D00269, M23598, Y00414, X)5290, SEG_HUMTHA0, M23597, M18116, M18115, M24788, M24791, M24789, M24787, M17589, M20912, M20911, M93281) (Egeland et al., 1987), dopamine D2 receptor (GenBank Accession No. AF050737), gamma-aminobutyric acid receptor (GenBank Accession No.s U47334, AF061786, AF061785, AA984687, AA776176, AA120821, AA663568, X55438, AA102670, X15376, X14767, X14766, X13584, T48783, L08485, M93435, M62400, SEG_HUMGABRB1, M59216, M59215, M59214, M59213, M59212) glutamate receptor (GenBank Accession No.s Q61625, A1200330, P42262, Q63225, A1160594, Q61605, A1205131, Q14832, U16126, AF009014, Q62643, P42263, Q14832, Q01812, Q90378, AA984569, AA984156, Z83848, AA947914, AA931601, Q61605, P39086, P41594, AA663711, U31216, U31215, L35318, A38485, JH0589, U10301, E99039, G1197726, U82083, U95025, E114243, P48058, A46212, P41594, D28538, D28539, M64752, U92459, U92458, U92457, U70870, X94552, S69349, S64316, S40369, L76631, L76627, X77748, X80818, X64830, X64829, X82068, X58633, U16129, U16125) and norepinephrine receptor (GenBank Accession No. M80776) (Coon et al., 1993). Genes associated with the liver type phosphofructokinase locus are of interest as possible candidate genes (Straub et al., 1994), as is the dopamine beta hydroxylase gene (GenBank Accession No. Y00096) (Sherrington et al., 1994). Based on current knowledge, it is likely that no one gene is responsible for manic depression in all cases. Rather, it is most likely a polygenic disease existing in different forms, depending upon the exact genotype of the individual. It should be understood that polymorphisms in any of the above genes, or other genes or loci may be involved in manic depression or a predisposition to it.

Anxiety Neurosis (Panic Disorder)

Anxiety neurosis is another disease which clearly has a genetic component. Pauls et al. (1980) analyzed 19 kindreds with anxiety neurosis and concluded that the segregation suggested autosomal dominant inheritance. Seven of the 19 kindreds were ascertained through a proband who had mitral valve prolapse in addition to anxiety neurosis. Autosomal dominant inheritance was equally supported by the other 12 pedigrees.

Transporter-facilitated uptake of serotonin has been implicated in anxiety in humans and in animal models and is the site of action of widely-used uptake-inhibiting antidepressant and antianxiety drugs. Lesch et al. (1996) found that transcription of the gene for serotonin transporter, termed SLC6A4 in rats and 5-HTT in humans (GenBank Accession No.s AF072904, Y13147, L05568), is modulated by a common polymorphism in its upstream regulatory region. They found that the short variant of the polymorphism reduces the transcriptional efficiency of the SLC6A4 gene promoter, resulting in decreased serotonin transporter expression and serotonin uptake in lymphoblasts. Overall, however, the associations of disease with the SLC6A4/5-HTT polymorphism represents only a small contribution to the genetic component of anxiety-related traits. Association studies in 2 independent samples totaling 505 subjects revealed that the promoter polymorphism of the transporter gene accounts for 3% to 4% of total variation and 7% to 9% of inherited variance of anxiety-related personality traits in individuals as well as sibships. This indicates that there are as yet undetermined genes which will contribute to the familial expression patterns of this disorder.

Schizophrenia

Schizophrenia is a common disorder with a lifetime prevalence of approximately 1%. The illness often develops in young adults who were previously normal, and is characterized by a constellation of symptoms including hallucinations and delusions (psychotic symptoms) and symptoms such as severely inappropriate emotional responses, disordered thinking and concentration, erratic behavior, as well as social and occupational deterioration.

Diagnosis of schizophrenia can be difficult. There is no universally accepted definition of the disorder, perhaps principally because there is no central feature, such as mood change in manic depression, and no characteristic pathology, such as neurofibrillary tangles in Alzheimer disease. The disease may not be a single entity.

The existence of a locus on the long arm of chromosome 5 that contributes to the etiology of schizophrenia was suggested by Bassett et al. (1988), who described 2 members of a family from Vancouver, an uncle and nephew, both of whom were schizophrenic at an early age and were partially trisomic for chromosome Sq11.2-q13.3.

Following the lead provided by the Vancouver family, other linkage studies were performed using DNA markers from 5q, including the gene for glucocorticoid receptor (GenBank Accession Nos. M10901 and M11050), which is a possible candidate for a schizophrenia-susceptibility gene because perturbations in glucocorticoid metabolism can induce psychotic symptoms. In a study of a single, well-documented kindred in a geographic isolate located above the Arctic circle, Kennedy et al. (1988) found no evidence of linkage to chromosome 5 markers. As pointed out by Lander (1988), the findings are consistent with the existence of 2 genetic types of schizophrenia.

Although the importance of genetic factors and the distinctness from manic-depressive psychosis are indicated by twin studies, the mode of inheritance is unclear. A priori, polygenic inheritance seems most likely, according to the rule that relatively frequent disorders such as this do not have simple monomeric genetic determination. Within the larger group there may be entities that behave in a simple mendelian manner, however.

Subsequent to these studies, several genes associated with schizophrenia and several other loci associated with schizophrenia susceptibility have been identified. A defect in the CHRNA7 gene, encoding the nicotinic cholinergic receptor alpha polypeptide 7 (GenBank Accession No. X70297) and located on chromosome 5 at q14, is likely involved in schizophrenia. The DRD3 gene, encoding dopamine receptor D3 (GenBank Accession No. U25441), located on chromosome 3 at ql3.3, is also likely important in schizophrenia susceptibility. In a study of ApoE (GenBank Accession Nos. U35114, M12529, X00199, U32510, and K00396) genotypes in schizophrenic patients coming to autopsy, Harrington et al. (1995) found that schizophrenia is associated with an increased E4 allele frequency. Other loci known to bear determinants related to schizophrenia susceptibility include 5q11.2-q13.3 (termed SCZD1), 6p23 (termed SCZD3), 22q11-q13.3 (termed SCZD4), and the less well defined loci termed SCZD2, SCZD5, SCZD6 and SCZD7. Because schizophrenia is a polygenic disease, the impact of polymorphisms existing in the genes is likely to be quite strong. That is, there are more possibilities for varied combinations of polymorphic genotypes, which may be reflected, for example, in the relative severity of the disease or in the response of the disease to various drug treatments among individuals.

Cystic Fibrosis

Cystic fibrosis (CF) is an inherited disorder affecting almost 30,000 people in North America. A defect in a single gene, encoding the cystic fibrosis transmembrane regulator (CFTR) is responsible for the disease. The defect in the chloride ion channel protein encoded by CFTR results in the production of excessively thick mucus in affected individuals. CFTR is also involved in other transport pathways, such that affected individuals have abnormally low levels of pancreatic enzymes, often resulting in malnutrition.

Cloning of the CF gene revealed that it encompasses around 250,000 bases (GenBank Accession No. AC000061). Kerem et al., (1989, Science 245:1073) found that approximately 70% of the mutations in CF patients correspond to a specific deletion of 3 bp, which results in the loss of a phenylalanine residue at amino acid position 508 (F508) of the product of the CF gene. Haplotype data based on DNA markers closely linked to the putative disease gene locus suggested that the remainder of the CF mutant gene pool consists of multiple, different mutations. Aside from the F508 mutation, there have indeed been multiple other mutations identified in the gene, including numerous single base pair alterations. The exact disease phenotype and severity of disease varies dramatically relative to the site(s) affected in an individual, indicating that many, if not most mutations lead to only partial inactivation of the gene product.

Sickle Cell Anemia

Sickle cell anemia (SCA) is a disease in which an abnormal beta globin protein results in a characteristic sickle-shaped red blood cell phenotype. The defect causes pain, acute chest syndrome, priapism, and abnormal red blood cell adhesion to the vascular endothelium.

Complications of the defect include damage to the spleen, avascular necrosis of bones, increased risk of stroke, retinal and kidney damage, among others.

The beta-thalassemias were among the first human genetic diseases to be examined by means of recombinant DNA analysis. In general, the molecular pathology of disorders resulting from mutations in the beta-globin gene region is the best known, this elucidation having started with SCA in the late 1940s. There are multiple molecular defects identified in the thalassemias, including deletion of portions of the gene, premature chain termination, point mutations in introns and splice junctions, frame shift mutations, fusion with other genes, and mutations resulting in single amino acid changes (reviewed by Steinberg & Adams, 1982, Am. J. Hemat., 12:81). The specific defect in SCA is a single base change resulting in conversion of a glutamic acid residue to valine (glu6-val). The severity of the disease ranges widely, indicating that variability in other genetic domains plays a role in the disease phenotype.

Gaucher Disease

Gaucher disease is the result of glucocerebrosidase deficiency, and is characterized by hematologic abnormalities with hypersplenism, bone lesions, skin pigmentationm and pingueculae. Gaucher disease is manifested in three primary forms, termed Types I (adult non-nuerologic form), II (infantile neurologic form) and III (adult neurologic form), and subtypes differing in various characteristics are known. The three different forms all appear to be the result of mutations to the same gene encoding glucocerebrosidase (acid beta glucosidase, GBA, GenBank Accession No. J03059), resulting in different catalytic efficiencies. It is likely that the different subtypes of the disease are the result of genetic heterogeneity, both within and outside of the GBA locus, among those affected by the mutations to the GBA gene.

Additional Examples of Diseases or Disease Susceptibilities According to the Invention

Abetalipoproteinemia

Adams-Stokes syndrome

Adenocarcinoma, kidney and small intestine

Adrenocortical hyperfunction

Agammaglobulinemia

Albright's syndrome

Alkaptonuria

Amyloidosis

Amyotrophic lateral sclerosis

Anorexia nervosa

Anxiety disorders

Becker's muscular dystrophy

Bipolar disorders

Blood diseases

Borderline personality

Brain stem glioma

Budd-Chiari syndrome

Cachexia, hypopituitary

Cardiac arrhythmias

Cardiovascular disease

Celiac sprue

Charcot-Marie-Tooth disease

Cholesterolosis

Combined familiar hyperlipidemia

Cowden disease

Crigler-Najjar syndrome

Crohn's disease

Cystic disease of kidney

Cystic fibrosis

Degenerative arthritis

Depression

Duchenne muscular dystrophy

Dystrophy, muscular and myotonic

Endocrine deficiency, multiple

Endogenous unipolar disorder

Epilepsy

Erythroleukemia, acute

Familial lecithin-cholesterol acyltransferase deficiency

Fibrocystic disease

Friedreich's ataxia

Gastrointestinal polyposis syndromes

Gaucher's disease

Gilbert's syndrome

Glaucoma

Glucose-6-phosphate dehydrogenase deficiency

Graves' disease

Hemophilia

Hemophilia A

Hemophilia B

Hereditary metabolic diseases

Hodgkin's disease

Huntington's disease

11β-Hydroxylase deficiency

21-Hydroxylase deficiency

Hyperaldosteronism

Hyperbetalipoproteinemia

Hypercalcemia

Hyptertension

Immunodeficiency

Infertility

Invertase deficiency

Irritable bowel syndrome

Juvenile chronic arthritis

Klinefelter's syndrome

Laurence-Moon-Biedl syndrome

Lesch-Nyhan syndrome

Leukemias

Lupus erythematosus, systemic

Lymphoproliferative diseases

Macular degeneration

Male pattern baldness

Mammary carcinoma

Mania

Marfan's syndrome

Melanin disorders

Metabolic disorders, including, but not limited to carbohydrate disorders

Migraine

Milkman's syndrome

Multiple endocrine adenomatosis

Multiple endocrine deficiency

Multiple endocrine gland hyperplasia

Multiple sclerosis

Myocardial infarction

Myopathic disorders

Myotonic dystrophy

Nephritis, including but not limited to: anti-glomerular basement membrane, hereditary chronic and interstitial

Neuralgic amyotrophy

Neuroblastoma

Neurofibromatosis

Neuromyopathy

Niemann-Pick disease

Non-Hodgkin's lymphoma

Nonmetabolic bone disease

Nonproliferative retinopathy

Obesity

Obsessive compulsive disorders

Organic brain syndrome

Osteoarthritis

Osteoporosis

Pancreatitis, chronic relapsing

Panic disorder

Paranoid disorders

Paranoid personality

paranoid schizophrenia

Parkinsonism

Personality disorders

Phenylketonuria

Phobic disorders

Pituitary gland diseases

Polycystic kidneys

Polycystic ovary syndrome

Polyglandular autoimmune syndrome

Prader-Willi syndrome

Prostate gland diseases, including, but not limited to, benign hyperplasia and carcinoma

Pulmonary system diseases

Renal disease(s)

Respiratory system diseases

Rheumatoid disease

Schizoaffective disorders

Schizophrenia

Schizophrenic disorders

Sclerosis, including but not limited to, amyotrophic lateral, primary lateral and progressive systemic

Sjögren's syndrome

Skin diseases

Sleep disorders

Tay-Sachs disease

Telangiectasia, hereditary hemorrhagic

Thalassemia, including but not limited to: alpha, major and minor

Thymoma

Trophoblastic neoplasms

Vascular insufficiency, mesenteric

Vasomotor disorders

Venous disease, degenerative and inflammatory

Waldenström's macroglobulinemia

Waterhouse-Friderichsen syndrome

Zollinger-Ellison syndrome.

Diseases Known to be Associated with Differential Genomic Representation According to the Invention

DNA re-arrangements known to be associated with disease are catalogued in McKusick, V. A.: Mendelian Inheritance in Man. Catalogs of Human Genes and Genetic Disorders. Baltimore: Johns Hopkins University Press, 1998 (12th edition). Non-limiting examples of diseases known to be associated with microdeletions (less than 25 kb deleted) include Fabry disease, Duchenne muscular dystrophy, Long QT syndrome, Angelman syndrome, adenomatous polyposis of the colon, retinoblastoma, neurofibromatosis type I, familial hypercholesterolemia, ataxia telangiectasia, Wilms tumor, Von Hippel-Lindau syndrome, Familial type 3 Alzheimer's disease, Tay-Sachs disease, and congenital adrenal hyperplasia due to 21-hydroxylase deficiency.

Non-limiting examples of diseases known to be associated with micro-duplications or amplifications include Duchenne muscular dystrophy, adenomatous polyposis of the colon, retinoblastoma, breast cancer, type 1, hemophilia A, neurofibromatosis, type I, Angelman syndrome, hermansky-pudlak syndrome, some testicular tumors, X-linked hyperphosphatemia, type II syndactyly, dystrophia myotonica 1, pseudovitamin D deficiency rickets, fragile site mental retardation and glioma of the brain.

Non-limiting examples of diseases known to be associated with genomic insertions include Tay-Sachs disease, ataxia telangiectasia, Bloom syndrome, adenomatous polyposis of the colon, dystrophia myotonica, xeroderma pigmentosum (complementation group A), fragile site mental retardation, type 2 non-polyposis familial colon cancer, congenital adrenal hyperplasia due to 21 hydroxylase deficiency, hyperlipoproteinemia type I, Bruton agammaglobulinemia, granulomatous disease, Complementation group A Fanconi anemia, multiple endocrine neoplasia and homocysteinuria.

This Mendelian Inheritance in Man compilation of genes and DNA sequences involved in human disorders is also available and continually updated online as the Online Mendelian Inheritance in Man database on the World Wide Web (ncbi.nlm.nih.gov/omim). Database accession numbers for the diseases or disorders listed above are provided below.

Microdeletions (less than 25 KB)

OMIM Disease Name

No.

301500 FABRY DISEASE

*192500 LONG QT SYNDROME 1

#105830 ANGELMAN SYNDROME; AS

*300377 DYSTROPHIN; DMD

*175100 ADENOMATOUS POLYPOSIS OF THE COLON; APC

*180200 RETINOBLASTOMA; RB1

*162200 NEUROFIBROMATOSIS, TYPE I; NF1

*606945 LOW DENSITY LIPOPROTEIN RECEPTOR; LDLR

*208900 ATAXIA-TELANGIECTASIA; AT

*194070 WILMS TUMOR 1; WT1

*193300 VON HIPPEL-LINDAU SYNDROME; VHL

*104311 ALZHEIMER DISEASE, FAMILIAL, TYPE 3

*147440 INSULIN-LIKE GROWTH FACTOR I; IGF1

#272800 TAY-SACHS DISEASE; TSD

*603372 THYROID-STIMULATING HORMONE RECEPTOR; TSHR

*201910 ADRENAL HYPERPLASIA, CONGENITAL, DUE TO 21-HYDROXYLASE DEFICIENCY

Micro-Duplications and Amplifications

OMIM Disease Name

No.

*300377 DYSTROPHIN; DMD

*175100 ADENOMATOUS POLYPOSIS OF THE COLON; APC

*180200 RETINOBLASTOMA; RB1

*113705 BREAST CANCER, TYPE 1; BRCA1

*306700 HEMOPHILIA A

*162200 NEUROFIBROMATOSIS, TYPE I; NF1

#105830 ANGELMAN SYNDROME; AS

*120150 COLLAGEN, TYPE I, ALPHA-1; COL1A1

#203300 HERMANSKY-PUDLAK SYNDROME; HPS

*273300 TESTICULAR TUMORS

*307800 HYPOPHOSPHATEMIA, X-LINKED

#186000 SYNDACTYLY, TYPE II

*264700 PSEUDOVITAMIN D DEFICIENCY RICKETS

#160900 DYSTROPHIA MYOTONICA 1

*309550 FRAGILE SITE MENTAL RETARDATION 1; FMR1

*137800 GLIOMA OF BRAIN

Insertions

OMIM Disease Name

No.

#272800 TAY-SACHS DISEASE; TSD

*208900 ATAXIA-TELANGIECTASIA; AT

#210900 BLOOM SYNDROME; BLM

*175100 ADENOMATOUS POLYPOSIS OF THE COLON; APC

#160900 DYSTROPHIA MYOTONICA 1

*278700 XERODERMA PIGMENTOSUM, COMPLEMENTATION GROUP A; XPA

*309550 FRAGILE SITE MENTAL RETARDATION 1; FMR1

#272800 TAY-SACHS DISEASE; TSD

*120436 COLON CANCER, FAMILIAL NONPOLYPOSIS, TYPE 2

*201910 ADRENAL HYPERPLASIA, CONGENITAL, DUE TO 21-HYDROXYLASE DEFICIENCY

*238600 HYPERLIPOPROTEINEMIA, TYPE I

*300300 BRUTON AGAMMAGLOBULINEMIA TYROSINE KINASE; BTK

*306400 GRANULOMATOUS DISEASE, CHRONIC; CGD

*227650 FANCONI ANEMIA, COMPLEMENTATION GROUP A; FANCA

*131100 MULTIPLE ENDOCRINE NEOPLASIA, TYPE I; MEN1

*236200 HOMOCYSTINURIA

Target DNA Sequences According to the Invention

The invention provides screening assays for the rapid detection of DNA rearrangements in test genomic DNA samples. Detection of these DNA rearrangements is accomplished using tagged primers that anneal specifically to target DNA sequences. Target DNA sequences can be either of known sequence (elected DNA target sequences) or of arbitrary sequence i.e. randomly selected. In a preferred embodiment, target DNA sequences, according to the invention, are from 15 to 40 nucleotides in length and are selected based upon known or suspected relationships between specific mutations and cancer or precancer. In another embodiment, target DNA sequences are distributed randomly throughout the genome.

In one embodiment, target DNA sequences are repetitive DNA elements or a part thereof. In humans, at least five classes of repetitive DNA sequences have been identified:

Telomeric regions, typically 5,000-12,000 bp in length, that are located at the end of chromosomes and contain repeats of the sequence TTAGGG;

Subtelomeric regions are repetitive sequences interspersed in last 500,000 bp of non-repetitive DNA, some of the sequences chromosome specific;

Microsatellite repeats are dispersed in euchromatic arms of most chromosomes and contain most commonly GT 20-60 bp repeats (copy number ca. 100,000 in the human genome); minisatellite repeats are typically 200-5,000 bp in length and consist of repeating units of 30-35 bp in length that are variable in sequence except for a core sequence 10-15 bp;

Alu repeats are typically 300 bp long and occur once in every 330 bases in the human genome (approx. one million copies); and

Satellite (a, I, II, III) sequences are found at the centromere of all human chromosomes. Repeats are typically 1,000-5,000 bp long and contain alternating arrays of 17 and 25 bp sequence that are AT rich. Satellite I, II, III long repeats are located in heteromeric chromatin.

In another embodiment, target DNA sequences are pseudogenes or part thereof.

In another embodiment, target DNA sequences are transposable elements.

In another embodiment, target DNA sequences are retrotransposable elements.

In another embodiment, target DNA sequences comprise a minisatellite marker sequence or part thereof.

In another embodiment, target DNA sequences are arbitrary, randomly selected, genomic DNA sequences.

### EXAMPLES

### Example 1

Human breast cancer is frequently associated with amplification of the HER-2/neu gene (see, for example, Press et al., J. Clin. Oncol. 20: 3095-3105). In order to determine a difference in the representation of HER-2/neu according to the invention, the following steps are taken on two genomic DNA samples, one having known (i.e., normal or reference) HER-2/neu representation and the other having unknown HER-2/neu representation.

A. Primer Extension.

A primer extension reaction is performed using the following primers:

1) Upstream primer

5′-gttacaagattctcacacgctaagg CTGGAAGCCACAAGGTAAAC-3′ (tag is in lower case; region that hybridizes to the HER-2/neu elected or target DNA sequence);

2) Downstream primer

5′-agttggcgaagcagtcgctagaaga ACACCCTTTTAAGTCTCAGG-3′ (tag is in lower case; region that hybridizes to the HER-2/neu elected or target DNA sequence). These primer extension primers flank a region of HER-2/neu sequence comprising nucleotides 11-180 of the sequence at GenBank Accession No. AH002823, which encompasses part of the HER-2/neu promoter region.

The indicated primer extension primers are mixed with 1 μg of template genomic DNA from the individual to be tested, in 1×Pfu buffer (20 mM Tris-HCl, pH 8.8, 10 mM KCl, 10 mM (NH₄)₂SO₄, 2 mM MgSO₄, 0.1% Triton-X-100 and 0.1 mg/ml nuclease-free BSA) in a total volume of 50 μl. The mixture is heated to 94° C. for 2 minutes to separate strands, and slowly cooled to room temperature, to permit primer annealing. 1 μl (2.5 U/μl) of cloned Pfu polymerase plus 1.25 μl of each dNTP (final concentration 200 μM) is added, and the sample is incubated at 72° C. for 3 minutes. The sample is then cycled to 94° C. for 2 minutes, then 50° C. for 1 minute, and 72° C. for 3 minutes to generate a population of primer extension products with an upstream primer or its complement and a downstream primer or its complement.

Primer extension primers are removed by the addition of 20 U of E. coli Exonuclease I (ExoI; New England Biolabs) and incubation at 37° C. for 20 minutes. ExoI is then inactivated by incubation at 80° C. for 20 minutes.

B. Amplification:

After removal of primer extension primers, two amplification primers (40 pmol of each primer in 1×Pfu buffer, final volume 75 μl) are added as follows:

a) Upstream Primer: 5′-gttacaagat tctcacacgc taagg-3′

b) Downstream primer: 5′-R6G-agttggcgaagcagtcgctagaaga (distinguishably labeled with R6G rhodamine label)

Amplification is performed by adding 1 μl of fresh, cloned Pfu polymerase and cycling the reaction as follows: 35 cycles of 94° C. for 45 sec., 50° C. for 45 sec., and 72° C. for 2 min. After each cycle, or at any chosen interval, an aliquot (0.5 μl) is withdrawn and loaded onto a prepared capillary electrophoresis apparatus. Separation is initiated and conducted during the amplification regimen. Amplified primer extension products are detected by fluorescence after separation over the length of the capillary. The signal strength of each fragment can be plotted for each cycle, to generate an amplification profile.

Expected amplified products are 219 bp long (169 bp of HER-2/neu genomic sequence and 50 bp of tag (25 nt of tag at each end). The intensity of the signal from 219 bp fragment in the reference genomic DNA sample is compared with the intensity in the experimental or unknown sample, and a difference is indicative of a difference in HER-2/neu sequence representation. During the linear region of the exponential amplification profile, the difference between the signal intensities is proportional to the difference in the genomic representation. That is, a higher sequence copy number will be represented by a proportionally higher signal during the linear phase of the amplification, permitting a determination of the relative sequence copy number.

There are several ways to multiplex the determination of differences in sequence representation described in this example. In one method, the same upstream and downstream tag sequences are joined to sequences that hybridize to upstream and downstream regions, respectively, of a new target gene. The size of the new target amplification product (or products) is selected so as to differ from that of the first target, such that amplification using the same upstream and downstream tag sequences as amplification primers amplifies different sized products that are detected according to the migration of the detectable label.

In another way to multiplex, the same or common upstream tag sequence is used on a set of upstream primer extension primers that differ in their 3′ regions. The differing 3′ regions are specific for each of the different upstream target sequences. For this approach, a set of downstream primer extension primers is used that has a different tag sequence for each different target and a different corresponding 3′ region specific for each different downstream target sequence. Amplification is performed using the common upstream tag sequence as an upstream amplification primer, and differentially labeled downstream tag sequences for the downstream amplification primer. The different labels are detected, and the identity of the label corresponds to the identity of the given target sequence. This scheme can be easily further multiplexed by selecting differentially sized amplification targets—differentially sized bands bearing a given detectable label are identified on the basis of size and label.

In yet another way to multiplex, additional different upstream tag sequences can be included in the same primer extension and amplification reactions. The additional upstream sequences will correspond to additional targets that may or may not generate differentially sized amplification products when paired with a set of differentially tagged, differentially labeled downstream primer extension and amplification primers. Again, the ability to detect both differently sized and differentially labeled products following amplification and capillary electrophoresis permits the identification and subsequent comparison of the signal from multiple targets with a much smaller number of primers than would otherwise be necessary. The use of common primers or limited numbers of primers for multiplex amplification has the benefit of reducing primer artifacts and increasing the uniformity of the amplification steps.

### Other Embodiments

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of determining, for a given genomic DNA sample, a variation in the representation of an elected target DNA sequence within said sample, said method comprising the steps of:
a) producing a labeled, amplified DNA fragment by subjecting a population of primer extension products to an amplification regimen wherein said population of primer extension products is generated from a nucleic acid sample comprising genomic DNA, wherein said population of primer extension products comprises a population of nucleic acid molecules comprising a first, upstream tag sequence or the complement thereof, an elected target DNA sequence or the complement thereof, and a second, downstream tag sequence or the complement thereof, wherein said amplification regimen is performed using an upstream amplification primer comprising said upstream tag sequence, and a labeled downstream amplification primer comprising said downstream tag sequence; and
b) detecting a difference in the amount of signal from the resulting labeled amplified DNA fragment relative to a reference,
wherein said difference in the signal is indicative of a variation in the representation of the elected target DNA sequence within said genomic DNA sample.

2. The method of claim 1, wherein said label is a fluorescent label.

3. The method of claim 1, wherein said step (b) comprises separating nucleic acid molecules made during said amplification regimen by size.

4. The method of claim 3, wherein said separating comprises capillary electrophoresis.

5. The method of claim 1, wherein said amplification regimen comprises at least two amplification reaction cycles, wherein each cycle comprises polymerase extension of annealed primers.

6. The method of claim 5 wherein said amplification regimen further comprises the steps, before said primer extension of annealed primers, of: 1) nucleic acid strand separation; and 2) oligonucleotide primer annealing.

7. The method of claim 6 further comprising the steps, during said amplification regimen and after at least one of said reaction cycles, of removing an aliquot of said amplification reaction, separating nucleic acid molecules by size and detecting the incorporation of said label wherein said detection determines the representation of the elected target DNA sequence within said genomic DNA sample.

8. The method of claim 1, further comprising, before step (a), the steps of:
1) subjecting a genomic DNA sample to a primer extension reaction, wherein said primer extension reaction is performed using:
i) an upstream primer extension primer comprising said first, upstream tag sequence and a covalently linked hybridization region that can anneal to a sequence comprised by said elected DNA sequence; and
ii) a downstream primer extension primer comprising said second, downstream tag sequence and a covalently linked hybridization region that can specifically anneal to a sequence comprised by said elected target DNA sequence in said genomic DNA sample, wherein said hybridization region of said downstream primer anneals on the opposite strand from and downstream of said upstream primer on said elected target DNA sequence; and
2) repeating step (1) to generate a population of primer extension products comprising both an upstream primer sequence or the complement thereof and a downstream primer sequence or the complement thereof.

9. The method of claim 8, further comprising the step, after step (2), of removing unincorporated upstream and downstream primers.

10. The method of claim 7 further comprising the step of sequencing said labeled amplified DNA fragment.

11. The method of claim 7, wherein said removing, separating and detecting are performed after each cycle in said regimen.

12. The method of claim 7, wherein said separating comprises capillary electrophoresis.

13. The method of claim 1, wherein said method is performed in a modular apparatus comprising a thermal cycler, a sampling device, a capillary electrophoresis device and a fluorescence detector.

14. The method of claim 1, wherein said tag sequences comprise 15 to 40 nucleotides.

15. The method of claim 9, wherein said step of removing unincorporated upstream and downstream amplification primers comprises degrading said primers.

16. The method of claim 15, wherein said degrading is performed using a heat labile exonuclease.

17. The method of claim 16, wherein said heat labile exonuclease is selected from the group consisting of Exonuclease I and Exonuclease VII.

18. The method of claim 17, wherein said heat labile exonuclease is thermally inactivated before continuing to step (d).

19. The method of claim 8, wherein either the upstream or downstream primer extension primer anneals to a repetitive DNA element.

20. The method of claim 8, wherein either the upstream or downstream primer extension primer anneals to a transposable DNA element.

21. The method of claim 1, wherein an increase of two fold or more in the said signal, relative to a reference, is indicative of a variation in the representation of the elected target DNA sequence within said genomic DNA sample.

22. The method of claim 1, wherein a decrease of two fold or more in the said signal, relative to a reference, is indicative of a variation in the representation of the elected target DNA sequence within said genomic DNA sample.

23. A method of determining, for a given genomic DNA sample, a variation in the representation of a group of elected target DNA sequences relative to a reference genomic DNA sample, said method comprising the steps of:
a) producing a set of labeled, amplified DNA fragments by subjecting a population of primer extension products generated from a nucleic acid sample comprising genomic DNA to an amplification regimen, wherein said population of primer extension products comprises a population of nucleic acid molecules comprising a common upstream tag sequence or the complement thereof, a member of said group of elected target DNA sequences, and a member of a set of downstream tag sequences or the complement thereof, wherein said amplification regimen is performed using:
i) an upstream amplification primer comprising said common upstream tag sequence; and
ii) a set of distinguishably labeled downstream amplification primers, each member of said set of labeled downstream amplification primers comprising a tag sequence comprised by a member of said set of downstream tag sequences, wherein each of said downstream tag sequences specifically corresponds to one member of said group of elected target DNA sequences; and
b) detecting a difference in the amount of signal from a labeled amplified fragment relative to said reference;
wherein a difference in the signal is indicative of a variation in the representation within said genomic DNA sample, of an elected target DNA sequence in said group of elected target DNA sequences.

24. The method of claim 23, further comprising, before step (a), the steps of:
1) subjecting a genomic DNA sample to a primer extension reaction, wherein said primer extension reaction is performed using:
i) a set of upstream primer extension primers, each member of said set comprising a sequence that can anneal to a sequence comprised by a member of said group of elected DNA elected target DNA sequences and said common upstream tag sequence; and
ii) a set of downstream primer extension primers, each member of said set of downstream primer extension primers comprising a region that can anneal to a sequence comprised by a member of said group of elected target DNA sequences and one of said set of corresponding downstream tag sequences, wherein said region that can anneal to a sequence comprised by a member of said group of elected target DNA sequences anneals downstream of and on the opposite strand from a member of said set of upstream primers;
2) repeating step (1) to generate a population of primer extension products comprising both an upstream primer sequence or the complement thereof and a downstream primer sequence or the complement thereof.

25. The method of claim 24, further comprising the step, after step (2), of removing unincorporated upstream and downstream primer extension primers.

26. The method of claim 23, wherein said distinguishable label is a fluorescent label.

27. The method of claim 23, wherein said step (b) comprises separating nucleic acid molecules made during said amplification regimen by size.

28. The method of claim 27, wherein said separating comprises capillary electrophoresis.

29. The method of claim 23, wherein said amplification regimen comprises at least two amplification reaction cycles, wherein each cycle comprises the step of polymerase extension of annealed primers.

30. The method of claim 29, wherein said amplification regimen further comprises, before said polymerase extension step, the steps of: 1) nucleic acid strand separation; and 2) oligonucleotide primer annealing.

31. The method of claim 23, further comprising the steps, during said amplification regimen and after at least one of said reaction cycles, of removing an aliquot of said amplification reaction, separating nucleic acid molecules by size, and detecting the incorporation of a said distinguishable label.

32. The method of claim 31, wherein said removing, separating and detecting are performed after each cycle in said regimen.

33. The method of claim 31, wherein said separating comprises capillary electrophoresis.

34. The method of claim 23, wherein said method is performed in a modular apparatus comprising a thermal cycler, a sampling device, a capillary electrophoresis device and a fluorescence detector.

35. The method of claim 23, wherein said tag sequences comprise 15 to 40 nucleotides.

36. The method of claim 25, wherein said step of removing unincorporated upstream and downstream amplification primers comprises degrading said primers.

37. The method of claim 36, wherein said degrading is performed using a heat labile exonuclease.

38. The method of claim 37, wherein said heat labile exonuclease is selected from the group consisting of Exonuclease I and Exonuclease VII.

39. The method of claim 37, wherein said heat labile exonuclease is thermally inactivated before continuing to step (d).

40. The method of claim 24, wherein either the upstream or downstream primer extension primer anneals to a repetitive DNA element.

41. The method of claim 24, wherein either the upstream or downstream primer extension primer anneals to a transposable DNA element.

42. The method of claim 23, wherein an increase of two fold or more in the said signal, relative to a reference, is indicative of said variation in the representation of the elected target DNA sequence within said genomic DNA sample.

43. The method of claim 23, wherein a decrease of two fold or more in the said signal, relative to a reference, is indicative of said variation in the representation of the elected target DNA sequence within said genomic DNA sample.

44. The method of claim 23, wherein each said upstream primer extension primer anneals to a sequence, comprised by a member of said group of elected target DNA sequences, that is located at a distance from a said downstream primer extension primer that is characteristic for said member of said group of elected target DNA sequences.

45. A method of determining, for a given genomic DNA sample, a variation in the representation of an arbitrary genomic DNA sequence to be interrogated within said sample, said method comprising the steps of:
a) producing a labeled, amplified DNA fragment by subjecting a population of primer extension products generated from a nucleic acid sample comprising genomic DNA to an amplification regimen, wherein said population of primer extension products comprises a population of nucleic acid molecules, each member of said population of nucleic acid molecules comprising an upstream tag sequence or the complement thereof, an arbitrary genomic DNA sequence and a downstream tag sequence or the complement thereof, wherein said amplification regimen is performed using:
i) an upstream amplification primer comprising said upstream tag sequence; and
ii) a distinguishably labeled downstream amplification primer comprising said downstream tag sequence; and
b) detecting a difference in the signal from the resulting labeled amplified DNA fragment relative to a reference;
wherein said difference in the signal is indicative of a variation in the representation of said arbitrary genomic DNA sequence within said genomic DNA sample.

46. The method of claim 45, further comprising the steps, before step (a), of:
1) subjecting a sample comprising genomic DNA to a primer extension reaction, wherein said primer extension reaction is performed using:
i) an upstream primer extension primer comprising a first arbitrary DNA sequence and said upstream tag sequence; and
ii) a downstream primer extension primer comprising a second arbitrary DNA sequence and said downstream tag sequence; and
2) repeating step (1) to generate a population of primer extension products comprising both an upstream tag sequence or the complement thereof and a downstream tag sequence or the complement thereof.

47. The method of claim 46, further comprising the step, after step (2), of removing unincorporated upstream and downstream primer extension primers.

48. The method of claim 45, wherein said label is a fluorescent label.

49. The method of claim 45, wherein said step (b) comprises separating nucleic acid molecules made during said amplification regimen by size and/or by charge.

50. The method of claim 49, wherein said separating comprises capillary electrophoresis.

51. The method of claim 45, wherein said amplification regimen comprises at least two amplification reaction cycles, wherein each cycle comprises the step of polymerase extension of annealed primers.

52. The method of claim 51 wherein said amplification regimen further comprises, before said step of polymerase extension of annealed primers, the steps of: i) nucleic acid strand separation; and ii) oligonucleotide primer annealing.

53. The method of claim 45, further comprising the steps, during said amplification regimen and after at least one of said reaction cycles, of removing an aliquot of said amplification reaction, separating nucleic acid molecules by size, and detecting the incorporation of a said distinguishable label.

54. The method of claim 53 further comprising the step, after said detecting the incorporation of a said distinguishable of sequencing the resulting amplified genomic DNA, wherein said sequencing determines the identity of said elected DNA sequence.

55. The method of claim 53, wherein said removing, separating and detecting are performed after each cycle in said regimen.

56. The method of claim 53, wherein said separating comprises capillary electrophoresis.

57. The method of claim 45, wherein said method is performed in a modular apparatus comprising a thermal cycler, a sampling device, a capillary electrophoresis device and a fluorescence detector.

58. The method of claim 45, wherein said tag sequence comprises 15 to 40 nucleotides.

59. The method of claim 47, wherein said step of removing unincorporated upstream and downstream amplification primers comprises degrading said primers.

60. The method of claim 59, wherein said degrading is performed using a heat labile exonuclease.

61. The method of claim 60, wherein said heat labile exonuclease is selected from the group consisting of Exonuclease I and Exonuclease VII.

62. The method of claim 60, wherein said heat labile exonuclease is thermally inactivated after degrading said upstream and downstream primer extension primers.

63. The method of claim 45, wherein an increase of two fold or more in the said signal, relative to a reference, is indicative of said variation in the representation of the arbitrary target DNA sequence within said genomic DNA sample.

64. The method of claim 45, wherein a decrease of two fold or more in the said signal, relative to a reference, is indicative of said variation in the representation of the arbitrary target DNA sequence within said genomic DNA sample.

65. A method of determining, for a given genomic DNA sample, a variation in the representation of one or more of a group of arbitrary genomic DNA sequences relative to a reference genomic DNA sample, said method comprising the steps of:
a) producing a set of labeled, amplified DNA fragments by subjecting a population of primer extension products generated from a sample comprising genomic DNA to an amplification regimen, wherein said population of primer extension products comprises a population of nucleic acid molecules comprising a common upstream tag sequence or the complement thereof, an arbitrary genomic DNA sequence, and a member of a set of downstream tag sequences or the complement thereof, wherein said amplification regimen is performed using:
i) an upstream amplification primer comprising said common upstream tag sequence; and
ii) a set of distinguishably labeled downstream amplification primers, each member of said set of labeled downstream amplification primers comprising a tag sequence comprised by a member of said set of downstream tag sequences; and
b) detecting a difference in the signal from a resulting labeled amplified fragment relative to said reference;
wherein a difference in the signal is indicative of a variation in the representation of an elected target DNA sequence, selected from the said group of arbitrary target DNA sequences, within said genomic DNA sample.

66. The method of claim 65, further comprising the steps, before step (a), of:
1) subjecting a genomic DNA sample to a primer extension reaction, wherein said primer extension reaction is performed using:
i) a set of upstream primer extension primers, each member of said set comprising said common upstream tag sequence and a region that can anneal to a member of said group of arbitrary genomic DNA sequences; and
ii) a set of downstream primer extension primers, each member of said set of downstream primer extension primers comprising a region that can anneal to a sequence comprised by a member of said group of arbitrary genomic DNA sequences and one of said set of downstream tag sequences, wherein said region than can anneal to a sequence comprised by a member of said group of arbitrary target DNA sequences anneals downstream of and on the opposite strand from a member of said set of upstream primer extension primers; and
b) repeating step (a) to generate a population of primer extension products comprising both an upstream tag sequence or the complement thereof and a downstream tag sequence or the complement thereof.

67. The method of claim 66, further including the step, after step (b) or removing unincorporated upstream and downstream primer extension primers.

68. The method of claim 65, wherein said distinguishable label is a fluorescent label.

69. The method of claim 65, wherein said step (b) comprises separating nucleic acid molecules made during said amplification regimen by size.

70. The method of claim 69, wherein said separating comprises capillary electrophoresis.

71. The method of claim 65, wherein said amplification regimen comprises at least two amplification reaction cycles, wherein each cycle comprises the step of polymerase extension of annealed primers.

72. The method of claim 71 wherein said amplification regimen further comprises the steps, before said step of polymerase extension of annealed primers, of i) nucleic acid strand separation and ii) oligonucleotide primer annealing.

73. The method of claim 65, further comprising the steps, during said amplification regimen and after at least one of said reaction cycles, of removing an aliquot of said amplification reaction, separating nucleic acid molecules by size, and detecting the incorporation of a said distinguishable label, and sequencing said amplified genomic DNA wherein said sequencing determines the identity of said arbitrary DNA sequence.

74. The method of claim 73, wherein said removing, separating and detecting are performed after each cycle in said regimen.

75. The method of claim 74, further comprising the step, after said detecting, of sequencing the resulting amplified genomic DNA, wherein said sequencing determines the identity of a said arbitrary DNA sequence.

76. The method of claim 73, wherein said separating comprises capillary electrophoresis.

77. The method of claim 66, wherein said method is performed in a modular apparatus comprising a thermal cycler, a sampling device, a capillary electrophoresis device and a fluorescence detector.

78. The method of claim 66, wherein said tag sequence comprises 15 to 40 nucleotides.

79. The method of claim 67, wherein said step of removing unincorporated upstream and downstream amplification primers comprises degrading said primers.

80. The method of claim 79, wherein said degrading is performed using a heat labile exonuclease.

81. The method of claim 80, wherein said heat labile exonuclease is selected from the group consisting of Exonuclease I and Exonuclease VII.

82. The method of claim 89, wherein said heat labile exonuclease is thermally inactivated after said degrading.

83. The method of claim 65, wherein an increase of two fold or more in the said signal, relative to a reference, is indicative of said variation in the representation of the arbitrary target DNA sequence within said genomic DNA sample.

84. The method of claim 65, wherein a decrease of two fold or more in the said signal, relative to a reference, is indicative of said variation in the representation of the arbitrary target DNA sequence within said genomic DNA sample.

85. The method of claim 66, wherein each said upstream primer extension primer anneals to a sequence, comprised by a member of said group of arbitrary DNA e sequences, that is located at a distance from said set of downstream primer extension primers that is characteristic for said upstream primer extension primer and said downstream primer extension primer.

86. A kit for the determination of the variation in representation of an elected target DNA sequence within a genomic DNA sample, said kit comprising:
a) an upstream primer extension primer comprising an upstream tag sequence and a covalently linked hybridization region that can anneal to a sequence at a known distance upstream of the elected target DNA sequence; and
b) a downstream primer extension primer comprising a downstream tag sequence and a covalently linked hybridization region that can anneal to an elected target DNA sequence within said genomic DNA sample.

87. The kit of claim 82, further comprising an upstream tag-specific amplification primer and a labeled downstream tag-specific amplification primer.
